(19) United States
(12) Patent Application Publication
(10) Pub. No.: US 2026/0327275 A1
(43) Pub. Date: Oct. 22, 2026

(54) PRODUCTION OF NATURAL PEPTIDE SWEETENER

(71) Applicant: Conagen Inc., Bedford, MA (US)

(72) Inventors: Guohong Mao, Burlington, MA (US); Timothy Joseph Hanly, Waltham, MA (US); Oliver Yu, Lexington, MA (US)

(73) Assignee: Conagen Inc., Bedford, MA (US)

(21) Appl. No.: 19/073,706

(22) Filed: Mar. 7, 2025

Related U.S. Application Data
(63) Continuation of application No. PCT/US2023/073553, filed on Sep. 6, 2023.
(60) Provisional application No. 63/374,764, filed on Sep. 7, 2022.

(51) Int. Cl. C07K 14/43; A23L 2/60; A23L 27/30; C12N 15/81

(52) U.S. Cl. CPC C07K 14/43; A23L 2/60; A23L 27/31; A23L 27/36; C12N 15/815

(57) ABSTRACT

Provided herein are biosynthetic methods for producing thaumatin and related compositions. Uses (e.g., as sweeteners) of the thaumatin produced using the methods are also provided.

### RELATED APPLICATIONS

This application is a Continuation of International Patent Application Serial No. PCT/US2023/073553, filed Sep. 6, 2023 which claims the benefit under 35 U.S.C. § 119(e) to U.S. Provisional Application No. 63/374,764, filed on Sep. 7, 2022, both entitled “PRODUCTION OF NATURAL PEPTIDE SWEETENER”, the entire contents of each of which are incorporated herein by reference.

### REFERENCE TO AN ELECTRONIC SEQUENCE LISTING

The contents of the electronic sequence listing (C149770091US01-SEQ-VLJ.xml; Size: 102,259 bytes; and Date of Creation: Mar. 7, 2025) are herein incorporated by reference in its entirety.

### FIELD OF THE INVENTION

The field of the invention relates to methods and processes useful in the production of natural peptide sweeteners.

### BACKGROUND

Zero- or low-calorie sweetener or sugar substitutes that can be used in foods and/or beverages to replace or reduce high-calorie sweeteners and/or sugar content are desirable. Thaumatin protein was first isolated from the fruit of West African plant Thaumatococcus daniellii Benth and has been reported to be 100,000 times sweeter than sucrose and may be suitable for use as sweetener.

### SUMMARY

The present disclosure, in some instances, provides methods of producing thaumatin. The method comprises culturing a recombinant yeast cell comprising a polynucleotide encoding a polypeptide comprising tandem repeats of thaumatin.

In a first aspect of the present invention, each repeat of thaumatin comprises the amino acid sequence of SEQ ID NO: 1. In a second aspect of the present invention, each repeat of thaumatin comprises the amino acid sequence of SEQ ID NO: 3. In some instances, each thaumatin in the tandem repeats is separated by a spacer. In one example, the spacer comprises a protease cleavage site for a yeast protease. In some cases, the spacer comprises the amino acid sequence of any one of SEQ ID NOs: 39, 41, and 43. In representative instances, the polypeptide comprises 2-8 repeats of thaumatin.

The polypeptide may further comprise an N-terminal signal peptide, such as a yeast alpha mating factor signal peptide. In some cases, the yeast alpha mating factor signal peptide comprises the amino acid sequence of SEQ ID NO: 37. In some embodiments, the signal peptide comprises an Ost1 signal peptide. In further embodiments, the polypeptide comprises an Ost1 signal peptide for each repeat of thaumatin. In representative examples, the Ost1 comprises the amino acid sequence of SEQ ID NO: 5.

In some embodiments, the polypeptide comprises a 2A linker between signal peptide-thaumatin open reading frames. In representative examples, the 2A linker comprises the amino acid sequence of SEQ ID NO: 7.

The polynucleotide may be operably linked to a promoter. In some cases, the promoter is an AOX1 promoter. The polynucleotide may also be operably linked to a transcription terminator. In some cases, the transcription terminator is an AOX1 terminator.

In some instances, the polynucleotide is provided on a vector, optionally wherein the vector is a plasmid. The polynucleotide may be integrated into the genome of the recombinant yeast cell. In representative examples, the polynucleotide is integrated into a HIS4 locus of the genome of the recombinant yeast cell.

The recombinant yeast cell may further comprise one or more polynucleotides encoding one or more chaperones selected from: PDI1, HAC1, ERO1, ERO2, ERV1, ERV2, KAR2, SEC1, SLY1, and GPX1. In a number of instances, the recombinant yeast cell further comprises a polynucleotide encoding PDI1, a polynucleotide encoding HAC1, one or more polynucleotides encoding PD1, ERO1, and ERV2, one or more polynucleotides encoding PDI, ERV1, and ERO1, or polynucleotides encoding both PDI1 and KAR2. In some cases, the one or more polynucleotides encoding the one or more chaperones are provided on one or more vectors. The one or more polynucleotides encoding the one or more chaperones may be integrated into the genome of the recombinant yeast cell. In some cases, the one or more polynucleotides are integrated into a HIS4 locus of the genome of the recombinant yeast cell.

The recombinant yeast cell may further comprise one or more polynucleotides encoding one or more proteases selected from KEX1, KEX2, and Ste13. In some cases, the one or more polynucleotides encoding the one or more proteases are provided on one or more vectors. The one or more polynucleotides encoding the one or more proteases may be integrated into the genome of the recombinant yeast cell. In some cases, the one or more polynucleotides are integrated into a AOX1 locus of the genome of the recombinant yeast cell.

The yeast cell may be a Pichia pastoris cell. The method may further comprise isolating the thaumatin. In one example, the method comprises isolating thaumatin I. In another example, the method comprises isolating thaumatin II. In some embodiments, the method further comprises isolating both thaumatin I and thaumatin II.

Uses of thaumatin produced using the method described herein as a sweetener are also provided. For example, in a further aspect, the present disclosure provides compositions or consumable products comprising the thaumatin produced using the method described herein. In some examples, the composition or the consumable product further comprises a second sweetener. In some examples, the second sweetener is a rebaudioside. In some examples, the composition or the consumable product at least one additive is selected from the group consisting of a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

In some instances, the consumable product is selected from: a food product, a beverage product, a nutraceutical, a pharmaceutical, a dietary supplement, a dental hygienic composition, an edible gel composition, a cosmetic product and a tabletop flavoring. In some instances, the beverage product is selected from the group consisting of a carbonated beverage product and a non-carbonated beverage product. In some examples, the beverage product is selected from the group consisting of a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink.

Further provided herein are recombinant yeast cells comprising a polynucleotide encoding a polypeptide comprising tandem repeats of thaumatin. In some examples, the recombinant yeast cell comprises a polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100% identical to any one of SEQ ID NOs: 9, 11, 51, 53, 55 and 57.

Further provided herein are recombinant yeast cells comprising a polynucleotide encoding a polypeptide comprising tandem repeats of thaumatin. In some examples, the recombinant yeast cell comprises a polynucleotide comprising a nucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100% identical to any one of SEQ ID NOs: 10, 12, 52, 54, 56, and 58.

Further provided herein are polypeptides comprising any one of SEQ ID NOs: 9, 11, 51, 53, 55 and 57. In some examples, the polypeptides comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100% identical to any one of SEQ ID NOs: 9, 11, 51, 53, 55 and 57.

Further provided herein are polynucleotides comprising any one of SEQ ID NOs: 10, 12, 52, 54, 56, and 58. In some examples, the polynucleotides comprises a nucleotide sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and 100% identical to any one of SEQ ID NOs: 10, 12, 52, 54, 56, and 58.

While the disclosure is susceptible to various modifications and alternative forms, specific instances thereof are shown by way of example in the figures and will herein be described in detail. It should be understood, however, that the figures and detailed description presented herein are not intended to limit the disclosure to the particular instances disclosed, but on the contrary, the intention is to cover all modifications, equivalents, and alternatives falling within the spirit and scope of the present disclosure as defined by the appended claims.

Other features and advantages of this invention will become apparent in the following detailed description of preferred instances of this invention, taken with reference to the accompanying figures.

### BRIEF DESCRIPTION OF THE DRAWINGS

The accompanying figures are not intended to be drawn to scale. In the figures, each identical or nearly identical component that is illustrated in various figures is represented by a like numeral. For purposes of clarity, not every component may be labeled in every figure. In the figures:

FIG. 1 provides a schematic of the plasmid map of pHKA-ThmIx1. The plasmid is composed of 8,168 bp, and contains an AOX1 promoter, and an Ost1 signal peptide fused to Thaumatin.

FIG. 2 provides a schematic of the plasmid map of pHKA-ThmIx4. The plasmid is composed of 14,318 bp, and contains 4 copies of the AOX1 promoter, and an Ost1 signal peptide fused to Thaumatin.

FIG. 3 provides a schematic of the plasmid map of pHKA-ThmIIx1. The plasmid is composed of 8,168 bp, and contains an AOX1 promoter, and an Ost1 signal peptide fused to Thaumatin.

FIG. 4 provides a schematic of the plasmid map of pHKA-ThmIIx4. The plasmid is composed of 14,318 bp, and contains 4 copies of the AOX1 promoter, and an Ost1 signal peptide fused to Thaumatin.

FIG. 5 provides an SDS-PAGE analysis of medium samples from induced culture. Legend: M: standard ladder; 1: Thaumatin I can be expressed in ThmI engineered strain; 2: Thaumatin II can be expressed in ThmII engineered strain. Arrows show thaumatin I and II.

FIG. 6 provides an HPLC analysis. In panel A is the thaumatin standard; in panel B is the media sample of the ThmI strain; and in panel C is the media sample of the ThmII strain. Arrows indicate peaks corresponding to thaumatin I and II.

FIGS. 7A-7C provide an LC-MS analysis of thaumatin standard and samples. In FIG. 7A is the thaumatin I standard; in FIG. 7B is the sample from pHKA-ThmI strain; and in FIG. 7C is the sample from pHKA-ThmII strain.

FIG. 8 provides a schematic of the plasmid map of pHKA-Ost1-ThmI-2A. The plasmid is composed of 8,915 bp, and contains an AOX1 promoter, an Ost1 signal peptide fused to Thaumatin I, a 2A linker, and an Ost1 signal peptide fused to Thaumatin I.

FIG. 9 provides a schematic of the plasmid map of pHKA-Ost1-ThmII-2A. The plasmid is composed of 8,915 bp, and contains an AOX1 promoter, an Ost1 signal peptide fused to Thaumatin II, a 2A linker, and an Ost1 signal peptide fused to Thaumatin II.

FIG. 10 provides an SDS-PAGE analysis of medium samples from induced culture. Lanes 1-6 are biological replicates of GS115 expressing pHKA-Ost1-ThmII-2A; Lane M is the protein ladder.

FIG. 11 provides an LC-MS analysis of media sample from pHKA-Ost1-ThmII-2A strain.

FIG. 12 demonstrates that co-expression of Pp PDI1 or AtPDI1 in Thm II strain increased thaumatin II production. Control: Parent strain, Pp PDI1 3 and 5: individual colonies from transformation of pPICZ-Pp PDI1 into Thm II strain. AtPDI1-3 and 5: individual colonies from transformation of pPICZ-AtPDI1 into Thm II strain.

FIG. 13 demonstrates that co-expression of HAC1 in Thm II strain increased thaumatin production. Control: Parent strain; HAC1 colony no. 3 and HAC1 colony no. 4: individual colonies from transformation of pPICZ-HAC1 into Thm II strain.

FIG. 14 demonstrates that co-expression of PDI/ERO/ERV in ThmII strain increased thaumatin production. Control: parent strain, 4 and 5: individual colonies from transformation of pPICZ-PpPDI/PpERO1/PpERV2 into ThmII strain.

FIG. 15 demonstrates that co-expression of PDI1/KAR2 in ThmII strain increased thaumatin production. Control: average of 5 replicates of ThmII parent strain, PDI1+KAR2: average of five replicates of a colony from transformation of pPICZ-PpPDI1/PpKAR2 into ThmII strain.

FIG. 16 provides an SDS-PAGE analysis of ThmIx4 fermentation samples. Lanes 1 and 2: samples before methanol induction; lanes 3-11: samples after methanol induction, 22, 27, 47, 55, 72, 77, 95, 102, and 118 hr; M: protein ladder; lane 12: thaumatin standard. Arrow shows thaumatin I.

FIG. 17 provides an SDS-PAGE analysis of ThmIIx4 fermentation samples. Lane 1: thaumatin standard; M: protein ladder; lanes 2 and 3: samples before methanol induction; lanes 4-12: samples after methanol induction, 22, 27, 47, 55, 72, 77, 95, 102, and 118 hr. Arrow shows thaumatin II.

FIG. 18 provides a schematic of the plasmid map of pHKA-Ost1-ThmI-linker-2Ax2, wherein the linker sequence comprises a KR(EA)5 linker (SEQ ID NO: 43). The plasmid is composed of 8,954 bp, and contains an AOX1 promoter, an Ost1 signal peptide fused to Thaumatin I, a KR(EA)5 linker (SEQ ID NO: 43), a 2A linker, and an Ost1 signal peptide fused to Thaumatin I.

FIG. 19 provides a schematic of the plasmid map of pHKA-Ost1-ThmI-linker-2Ax8, wherein the linker sequence comprises a KR(EA)5 linker (SEQ ID NO: 43). The plasmid is composed of 13,652 bp, and contains an AOX1 promoter, an Ost1 signal peptide fused to Thaumatin I, and 7 repeats of a KR(EA)5 linker (SEQ ID NO: 43), a 2A linker, and an Ost1 signal peptide fused to Thaumatin I.

FIG. 20 provides a schematic of the plasmid map of pHKA-Ost1-ThmI-linker-2Ax2M8, wherein the linker sequence comprises a KR(EA)5 linker (SEQ ID NO: 43). The plasmid is composed of 28,806 bp, and contains 8 copies of an AOX1 promoter, an Ost1 signal peptide fused to Thaumatin I, a KR(EA)5 linker (SEQ ID NO: 43), an 2A linker, and an Ost1 signal peptide fused to Thaumatin I.

FIG. 21 provides a schematic of the plasmid map of pHKA-Ost1-ThmII-linker-2Ax2, wherein the linker sequence comprises a KR(EA)5 linker (SEQ ID NO: 43). The plasmid is composed of 8,954 bp, and contains an AOX1 promoter, an Ost1 signal peptide fused to Thaumatin II, a KR(EA)5 linker (SEQ ID NO: 43), an 2A linker, and an Ost1 signal peptide fused to Thaumatin II.

FIG. 22 provides a schematic of the plasmid map of pHKA-Ost1-ThmII-linker-2Ax8, wherein the linker sequence comprises a KR(EA)5 linker (SEQ ID NO: 43). The plasmid is composed of 13,652 bp, and contains an AOX1 promoter, an Ost1 signal peptide fused to Thaumatin II, and 7 repeats of a KR(EA)5 linker (SEQ ID NO: 43), an 2A linker, and an Ost1 signal peptide fused to Thaumatin II.

FIG. 23 provides a schematic of the plasmid map of pHKA-Ost1-ThmII-2Ax2M8. The plasmid is composed of 28,806 bp, and contains 8 copies of an AOX1 promoter, an Ost1 signal peptide fused to Thaumatin II, a KR(EA)5 linker (SEQ ID NO: 43), an 2A linker, and an Ost1 signal peptide fused to Thaumatin II.

FIG. 24 provides an SDS-PAGE analysis of medium samples from induced culture. Lanes 1-6 are biological replicates of GS115 expressing pHKA-Ost1-ThmII-linker-2Ax2, wherein the linker sequence comprises a KR(EA)5 linker (SEQ ID NO: 43); Lane M is the protein ladder.

### DEFINITIONS

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of ectopic proteins. It includes bacteria, yeast, plant cells and animal cells. It may include prokaryotic or eukaryotic host cells which are modified to express a recombinant protein and cultivated in an appropriate culture medium. It also includes the in vitro expression of proteins based on cellular components, such as ribosomes.

“Coding sequence” is to be given its ordinary and customary meaning to a person of ordinary skill in the art, and is used without limitation to refer to a DNA sequence that encodes for a specific amino acid sequence.

“Growing the Cellular System”. Growing includes providing an appropriate medium that would allow cells to multiply and divide, to form a cell culture. It also includes providing resources so that cells or cellular components can translate and make recombinant proteins.

“Protein Expression”. Protein production can occur after gene expression. It consists of the stages after DNA has been transcribed to messenger RNA (mRNA). The mRNA is then translated into polypeptide chains, which are ultimately folded into proteins. DNA or RNA may be present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

“Yeast”. According to the current disclosure a yeast are eukaryotic, single-celled microorganisms classified as members of the fungus kingdom. Yeasts are unicellular organisms which are believed to have evolved from multicellular ancestors.

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subject technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing a thaumatin composition.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polyaminoacid product. Thus, exemplary polypeptides include polyaminoacid products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super-families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. Typically, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the field, and is used without limitation to refer to the transfer of a polynucleotide into a target cell for further expression by that cell. The transferred polynucleotide can be incorporated into the genome or chromosomal DNA of a target cell, resulting in genetically stable inheritance, or it can replicate independent of the host chromosomal DNA. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### DETAILED DESCRIPTION

Thaumatin is a group of intensely sweet proteins which was first isolated from the fruit of plant Thaumatococcus daniellii Benth. found in West Africa. Thaumatin is 1600 times sweeter than sucrose on a weight basis or approximately 100,000 on a molar basis. There are two forms of thaumatin identified in fruit, thaumatin I and II. The molecular mass of the protein is 22 kDa. Thaumatin I has 207 amino acid residues. Thaumatin II is also composed of 207 amino acid residues but has differs in 4 amino acids from Thaumatin I. Thaumatin is heat-stable and its sweet taste is preserved after incubation at a pH below 5.5 for 1 hr. At these pH values the sweetener is stable during heat-intensive processing steps such as pasteurization, canning, baking, and ultra-high temperature processing. Above 70 °C at a pH of 7.0, loss of sweetness was observed. Thaumatin has eight intramolecular disulfide bonds which are believed to relate to its heat stability. Loss of sweetness can be associated with heat-driven denaturation or breakage of the disulfide bonds within the protein. In the present invention, engineered Pichia strains for producing and secreting thaumatin I and thaumatin II were engineered. The product thaumatin was characterized by the same taste as thaumatin extracted from fruit.

### Production of Thaumatin in Recombinant Yeast Cells

The present disclosure, in some instances, provide methods of producing thaumatin, in which multiple strategies are employed to increase thaumatin folding, secretion and/or production in engineered yeast cells (e.g., engineered Pichia cells).

In some instances, a method of producing thaumatin described herein comprises culturing a recombinant yeast cell comprising a polynucleotide encoding a fusion polypeptide which comprises tandem repeats of thaumatin. In some examples, the polynucleotide is provided on a vector (e.g., a plasmid such as an expression plasmid). In some cases, the plasmid is a high copy plasmid (e.g., for high-level expression of the fusion polypeptide comprising the tandem repeats of thaumatin). In some cases, the polynucleotide is integrated into the genome of the recombinant yeast cell. For example, in some embodiments, the polynucleotide is integrated into a HIS4 locus of the genome of the recombinant yeast cell.

In some instances, the method of producing thaumatin described herein comprises culturing a recombinant yeast cell comprising a polynucleotide encoding a fusion polypeptide which comprises tandem repeats of thaumatin. In some examples, the polynucleotide comprises 2-8 (2-20, 2-16, 2-15, 2-10, 2-8, 8-20, 8-16, 8-15, 8-10, 10-20, 10-16, 10-15, 15-20, 15-16, 16-20) repeats of thaumatin. In some examples, the polynucleotide comprises 2, 8, 10, 15, 16, 20 repeats of thaumatin. In some examples, the polynucleotide comprises 2 repeats of thaumatin. In some examples, the polynucleotide comprises 8 repeats of thaumatin. In some examples, the polynucleotide comprises 16 repeats of thaumatin.

In some instances, the method of producing thaumatin described herein comprises culturing a recombinant yeast cell comprising a polynucleotide encoding a fusion polypeptide. In some embodiments, the fusion polypeptide comprises an amino acid sequence at least 80% (at least 80%, at least 85%, at least 90%, at least 95%, at least 98%, at least 99%) identical to the amino acid sequence of any one of SEQ ID NOs: 9, 11, 51, 53, 55 and 57. In some embodiments, the fusion polypeptide comprises an amino acid sequence at least 80%, at least 85%, at least 90%, at least 95%, at least 98%, at least 99% identical to the amino acid sequence of any one of SEQ ID NOs: 9, 11, 51, 53, 55 and 57. In some embodiments, the fusion polypeptide comprises the amino acid sequence of any one of SEQ ID NOs: 9, 11, 51, 53, 55 and 57. In some instances, each repeat of thaumatin comprises an amino acid sequence that is at least 70% (e.g., at least 70%, at least 75%, at least 80%, at least 85%, at least 90%, at least 99%, at least 99%, or even 100%) identical to the amino acid sequence of SEQ ID NO: 1. In some examples, each repeat of thaumatin comprises the amino acid sequence of SEQ ID NO: 1. In some examples, each repeat of thaumatin comprises the amino acid sequence of SEQ ID NO: 3. More generally, each polypeptide comprises at least 2 (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20) repeats of thaumatin. In some examples, each polypeptide comprises 2-20 (e.g., 2-20, 2-15, 2-10, 2-5, 5-20, 5-15, 5-10, 8-16 10-20, 10-15, or 15-20) repeats of thaumatin. In some cases, each polypeptide comprises 2, 3, 4, 5, 6, 7, 8, 9, 10, 11, 12, 13, 14, 15, 16, 17, 18, 19, or 20 repeats of thaumatin. In some examples, each polypeptide comprises 2 repeats of thaumatin. In some examples, each polypeptide comprises 8 repeats of thaumatin. In some examples, each polypeptide comprises 16 repeats of thaumatin.

In representative instances, in the fusion polypeptide comprising tandem repeats of thaumatin, each thaumatin repeat is separated by a spacer. In some cases, the spacer is cleaved by a protease (e.g., cleaved in vivo by a protease in the yeast cell). As such, in some cases, each spacer between the thaumatin repeats comprises a protease cleavage site for a yeast protease. In some embodiments, each spacer comprises an amino acid sequence at least 70% (e.g., at least 70%, at least 75%, at least 80%, at least 85%, at least 90%, at least 95%, or at least 99%) identical to the amino acid sequence of any one of SEQ ID NOs: 39, 41, 43, 45, 47, or 49. In some examples, each spacer comprises the amino acid sequence of any one of SEQ ID NOs: 39, 41, 43, 45, 47, or 49.

In some examples, the fusion polypeptide further comprises an N-terminal signal peptide. A “signal peptide” refers to a short peptide present at the N-terminus of a protein destined to be secreted from a cell. In some cases, a signal peptide comprises a stretch of hydrophobic amino acid residues that facilitate the translocation of a newly synthesized peptide or protein to the cell membrane for subsequent secretion through the cell membrane. More typically, a signal peptide is 5-23 (e.g., 5, 6, 7, 8, 9, 10, 11, 12, 13, 14, 15, 16, 17, 18, 19, 20, 21, 22, or 23) amino acids in length. A protein with a signal peptide can be encapsulated in a secretory vesicle and trafficked to the cell membrane via the secretory pathway. The mechanism by which a newly synthesized peptide or protein comprising a signal peptide is secreted from the cell will be known by a person having ordinary skill in the art.

In some instances, the signal peptide is a yeast alpha mating factor signal peptide. In some examples, the yeast alpha mating factor signal peptide comprises the amino acid sequence of SEQ ID NO: 37. In other instances, the signal peptide is an Ost1 signal peptide. In some examples, the fusion polypeptide comprises an Ost1 signal peptide for each repeat of thaumatin. In some examples, the Ost1 comprises the amino acid sequence of SEQ ID NO: 5.

In some instances, the fusion polypeptide comprises a 2A linker between signal peptide-thaumatin open reading frames. In some examples, the 2A linker comprises the amino acid sequence of SEQ ID NO: 7.

In representative examples, the polynucleotide encoding the fusion polypeptide comprising the tandem repeats of thaumatin is operably linked to a promoter. In some cases, the promoter is a constitutive promoter (e.g., a constitutive promoter in yeast). In some cases, the promoter comprises an AOX1 promoter (e.g., a yeast AOX1 promoter). In some cases, the polynucleotide encoding the fusion polypeptide comprising the tandem repeats of thaumatin is operably linked to a transcription terminator. In some cases, the transcription terminator is an AOX1 terminator (e.g., a yeast AOX1 terminator).

### Co-Expression of Chaperones

In some examples, the method described herein comprises co-expressing the fusion polypeptide comprising the tandem repeats of thaumatin with one or more chaperones to facilitate intramolecular disulfide bond formation, folding, and/or secretion. In some cases, the one or more chaperones are selected from: PDI1, HAC1, ERO1, ERO2, ERV1, ERV2, KAR2, SEC1, SLY1, and GPX1. Chaperones of the same yeast strain as the yeast recombinant cell used for expression of the polypeptide may be used. Heterologous chaperones from other yeast strains may also be used. Non-limiting examples of chaperones and their Genbank accession numbers are provided below in Table 3. As such, in some cases, the yeast recombinant cell used in the methods described herein further comprises one or more polynucleotides encoding one or more chaperones selected from PDI1, HAC1, ERO1, ERO2, ERV1, ERV2, KAR2, SEC1, SLY1, and GPX1. PDI1 is the structural gene for Protein Disulfide Isomerase (PDI).

In some examples, the PDI1 comprises an amino acid sequence at least 70% (e.g., at least 70%, at least 75%, at least 80%, at least 85%, at least 90%, at least 99%, or even 100%) identical to the amino acid sequence of SEQ ID NO: 13. In some cases, the PDI comprises the amino acid sequence of SEQ ID NO: 13. In some examples, the polynucleotide encoding the PDI comprises a nucleotide sequence at least 70% (e.g., at least 70%, at least 75%, at least 80%, at least 85%, at least 90%, at least 95%, or at least 100%) identical to the nucleotide sequence of SEQ ID NO: 14. In some cases, the polynucleotide encoding the PDI1 comprises the nucleotide sequence of SEQ ID NO: 14.

In some examples, the PDI1 comprises an amino acid sequence at least 70% (e.g., at least 70%, at least 75%, at least 80%, at least 85%, at least 90%, at least 95%, or at least 99%) identical to the amino acid sequence of SEQ ID NO: 15. In some cases, the PDI1 comprises the amino acid sequence of SEQ ID NO: 15. In some examples, the polynucleotide encoding the PDI1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 16. In some cases, the polynucleotide encoding the PDI1 comprises the nucleotide sequence of SEQ ID NO: 16.

In some examples, the HAC1 comprises an amino acid sequence at least 70% (e.g., at least 70%, at least 75%, at least 80%, at least 85%, at least 90%, at least 95%, or at least 99%) identical to the amino acid sequence of SEQ ID NO: 17. In some cases, the HAC1 comprises the amino acid sequence of SEQ ID NO: 17. In some examples, the polynucleotide encoding the HAC1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 18. In some cases, the polynucleotide encoding the HAC1 comprises the nucleotide sequence of SEQ ID NO: 18.

In some examples, the ERO1 comprises an amino acid sequence at least 70% (e.g., at least 70%, at least 75%, at least 80%, at least 85%, at least 90%, at least 95%, or at least 99%) identical to the amino acid sequence of SEQ ID NO: 19 or SEQ ID NO: 27. In some cases, the ERO1 comprises the amino acid sequence of SEQ ID NO: 19 or SEQ ID NO: 27. In some examples, the polynucleotide encoding the ERO1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 20 or SEQ ID NO: 28. In some cases, the polynucleotide encoding the ERO1 comprises the nucleotide sequence of SEQ ID NO: 20 or SEQ ID NO: 28.

In some examples, the ERO2 comprises an amino acid sequence at least 70% (e.g., at least 70%, at least 75%, at least 80%, at least 85%, at least 90%, at least 95%, or at least 99%) identical to the amino acid sequence of SEQ ID NO: 21. In some cases, the ERO2 comprises the amino acid sequence of SEQ ID NO: 21. In some examples, the polynucleotide encoding the ERO2 comprises a nucleotide sequence at least 70% (e.g., at least 70%, at least 75%, at least 80%, at least 85%, at least 90%, at least 95%, or at least 99%) identical to the nucleotide sequence of SEQ ID NO: 22. In some cases, the polynucleotide encoding the ERO2 comprises the nucleotide sequence of SEQ ID NO: 22.

In some examples, the ERV1 comprises an amino acid sequence at least 70% (e.g., at least 70%, at least 75%, at least 80%, at least 85%, at least 90%, at least 95%, or at least 99%) identical to the amino acid sequence of SEQ ID NO: 23. In some cases, the ERV1 comprises the amino acid sequence of SEQ ID NO: 23. In some examples, the polynucleotide encoding the ERV1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 24. In some cases, the polynucleotide encoding the ERV1 comprises the nucleotide sequence of SEQ ID NO: 24.

In some examples, the ERV2 comprises an amino acid sequence at least 70% (e.g., at least 70%, at least 75%, at least 80%, at least 85%, at least 90%, at least 95%, or at least 99%) identical to the amino acid sequence of SEQ ID NO: 25. In some cases, the ERV2 comprises the amino acid sequence of SEQ ID NO: 25. In some examples, the polynucleotide encoding the ERV2 comprises a nucleotide sequence at least 70% (e.g., at least 70%, at least 75%, at least 80%, at least 85%, at least 90%, at least 95%, or at least 99%) identical to the nucleotide sequence of SEQ ID NO: 26. In some cases, the polynucleotide encoding the ERV2 comprises the nucleotide sequence of SEQ ID NO: 26.

In some examples, the KAR2 comprises an amino acid sequence at least 70% (e.g., at least 70%, at least 75%, at least 80%, at least 85%, at least 90%, at least 95%, or at least 99%) identical to the amino acid sequence of SEQ ID NO: 29. In some cases, the KAR2 comprises the amino acid sequence of SEQ ID NO: 29. In some examples, the polynucleotide encoding the KAR2 comprises a nucleotide sequence at least 70% (e.g., at least 70%, at least 75%, at least 80%, at least 85%, at least 90%, at least 95%, or at least 99%) identical to the nucleotide sequence of SEQ ID NO: 30. In some cases, the polynucleotide encoding the KAR2 comprises the nucleotide sequence of SEQ ID NO: 30.

In some examples, the SEC1 comprises an amino acid sequence at least 70% (e.g., at least 70%, at least 75%, at least 80%, at least 85%, at least 90%, at least 95%, or at least 99%) identical to the amino acid sequence of SEQ ID NO: 31. In some cases, the SEC1 comprises the amino acid sequence of SEQ ID NO: 31. In some examples, the polynucleotide encoding the SEC1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 32. In some cases, the polynucleotide encoding the SEC1 comprises the nucleotide sequence of SEQ ID NO: 32.

In some examples, the SLY1 comprises an amino acid sequence at least 70% (e.g., at least 70%, at least 75%, at least 80%, at least 85%, at least 90%, at least 99%, or at least 99%) identical to the amino acid sequence of SEQ ID NO: 33. In some cases, the SLY1 comprises the amino acid sequence of SEQ ID NO: 33. In some examples, the polynucleotide encoding the SLY1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 34. In some cases, the polynucleotide encoding the SLY1 comprises the nucleotide sequence of SEQ ID NO: 34.

In some examples, the GPX1 comprises an amino acid sequence at least 70% (e.g., at least 70%, at least 75%, at least 80%, at least 85%, at least 90%, at least 95%, or at least 99%) identical to the amino acid sequence of SEQ ID NO: 35. In some cases, the GPX1 comprises the amino acid sequence of SEQ ID NO: 35. In some examples, the polynucleotide encoding the GPX1 comprises a nucleotide sequence at least 70% (e.g., at least 70%, at least 75%, at least 80%, at least 85%, at least 90%, at least 95%, or at least 99%) identical to the nucleotide sequence of SEQ ID NO: 36. In some cases, the polynucleotide encoding the GPX1 comprises the nucleotide sequence of SEQ ID NO: 36.

In some examples, the recombinant yeast cell further comprises a polynucleotide encoding PDI1 (e.g., a PDI1 comprising the amino acid sequence of SEQ ID NO: 13). In some cases, the recombinant yeast cell further comprises a polynucleotide comprising the nucleotide sequence of SEQ ID NO: 14.

In some examples, the recombinant yeast cell further comprises a polynucleotide encoding PDI1 (e.g., a PDI1 comprising the amino acid sequence of SEQ ID NO: 15). In some cases, the recombinant yeast cell further comprises a polynucleotide comprising the nucleotide sequence of SEQ ID NO: 16.

In some examples, the recombinant yeast cell further comprises a polynucleotide encoding HAC1 (e.g., a HAC1 comprising the amino acid sequence of SEQ ID NO: 17). In some cases, the recombinant yeast cell further comprises a polynucleotide comprising the nucleotide sequence of SEQ ID NO: 18.

In some examples the recombinant yeast cell further comprises a polynucleotide encoding PDI1 (e.g., a PDI1 comprising the amino acid sequence of SEQ ID NO: 15), a polynucleotide encoding ERO1 (e.g., an ERO1 comprising the amino acid sequence of SEQ ID NO: 19), and a polynucleotide encoding ERV2 (e.g., an ERV2 comprising the amino acid sequence of SEQ ID No: 25). In some cases, the recombinant yeast cell further comprises a polynucleotide comprising the nucleotide sequence of SEQ ID NO: 16, a polynucleotide comprising the nucleotide sequence of SEQ ID NO: 20, and a polynucleotide comprising the nucleotide sequence of SEQ ID NO: 26.

In some examples, the recombinant yeast cell further comprises a polynucleotide encoding PDI1 (e.g., a PDI1 comprising the amino acid sequence of SEQ ID NO: 15), a polynucleotide encoding ERV1 (e.g., an ERV1 comprising the amino acid sequence of SEQ ID NO: 23), and a polynucleotide encoding ERO2 (e.g., an ERO2 comprising the amino acid sequence of SEQ ID No: 21). In some cases, the recombinant yeast cell further comprises a polynucleotide comprising the nucleotide sequence of SEQ ID NO: 16, a polynucleotide comprising the nucleotide sequence of SEQ ID NO: 24, and a polynucleotide comprising the nucleotide sequence of SEQ ID NO: 22.

In some examples, the recombinant yeast cell further comprises a polynucleotide encoding PDI1 (e.g., a PDI comprising the amino acid sequence of SEQ ID NO: 15), a polynucleotide encoding KAR2 (e.g., an KAR2 comprising the amino acid sequence of SEQ ID NO: 29). In some cases, the recombinant yeast cell further comprises a polynucleotide comprising the nucleotide sequence of SEQ ID NO: 16 and a polynucleotide comprising the nucleotide sequence of SEQ ID NO: 30.

In some examples, each of the one or more polynucleotides encoding the one or more chaperones is operably linked to a promoter (e.g., a promoter selected from AOX1 promoter, GAP1 promoter, and CAT1 promoter).

In some examples, the one or more polynucleotides encoding the one or more chaperones are provided on one or more vectors (e.g., plasmids). In some examples, the one or more (e.g., 1, 2, 3, 4, 5 or more) polynucleotides encoding the one or more chaperones are provided on the same vector as the polynucleotide encoding the polypeptide comprising the tandem thaumatin repeats. In some other examples, the one or more (e.g., 1, 2, 3, 4, 5 or more) polynucleotides encoding the one or more chaperones are provided on different vectors as the polynucleotide encoding the polypeptide comprising the tandem thaumatin repeats.

In some examples, the one or more polynucleotides encoding the one or more chaperones are integrated into the genome of the recombinant yeast cell. In some cases, the one or more polynucleotides are integrated into a HIS4 or AOX1 locus of the genome of the recombinant yeast cell.

In some embodiments, recombinant yeast cell comprising a polynucleotide encoding a fusion polypeptide comprising tandem repeats of thaumatin. In some embodiments, the thaumatin is selected from the group consisting of thaumatin I and thaumatin II.

In some embodiments, each repeat of thaumatin comprises an amino acid sequence at least 70% (e.g., at least 70%, at least 75%, at least 80%, at least 85%, at least 90%, at least 95%, or at least 99%) identical to the amino acid sequence of SEQ ID NO: 1. In some embodiments, each repeat of thaumatin comprises the amino acid sequence of SEQ ID NO: 1. In some embodiments, each repeat of thaumatin comprises an amino acid sequence at least 70% (e.g., at least 70%, at least 75%, at least 80%, at least 85%, at least 90%, at least 95%, or at least 99%) identical to the amino acid sequence of SEQ ID NO: 3. In some embodiments, each repeat of thaumatin comprises the amino acid sequence of SEQ ID NO: 3.

In some embodiments, each thaumatin in the tandem repeats is separated by a spacer. In some embodiments, the spacer comprises a protease cleavage site for a yeast protease. In some embodiments, the spacer comprises an amino acid sequence at least 70% (e.g., at least 70%, at least 75%, at least 80%, at least 85%, at least 90%, at least 95%, or at least 99%) identical to the amino acid sequence of any one of SEQ ID NOs: 39, 41, and 43. In some embodiments, the spacer comprises the amino acid sequence of any one of SEQ ID NOs: 39, 41, and 43.

In some embodiments, the fusion polypeptide comprises 2-8 repeats of thaumatin. In some embodiments, the fusion polypeptide comprises 2-8 (2-20, 2-16, 2-15, 2-10, 2-8, 8-20, 8-16, 8-15, 8-10, 10-20, 10-16, 10-15, 15-20, 15-16, 16-20) repeats of thaumatin. In some embodiments, the fusion polypeptide comprises 2, 8, 10, 15, 16, 20 repeats of thaumatin. In some embodiments, the fusion polypeptide comprises 2 repeats of thaumatin. In some embodiments, the fusion polypeptide comprises 8 repeats of thaumatin. In some embodiments, the fusion polypeptide comprises 16 repeats of thaumatin.

In some embodiments, the fusion polypeptide comprises an amino acid sequence at least 70% (e.g., at least 70%, at least 75%, at least 80%, at least 85%, at least 90%, at least 95%, or at least 99%) identical to the amino acid sequence of any one of SEQ ID NOs: 9, 11, 51, 53, 55 and 57. In some embodiments, the fusion polypeptide comprises an amino acid sequence at least 80% identical to the amino acid sequence of any one of SEQ ID NOs: 9, 11, 51, 53, 55 and 57. In some embodiments, the fusion polypeptide comprises an amino acid sequence of any one of SEQ ID NOs: 9, 11, 51, 53, 55, and 57.

In some embodiments, the polynucleotide comprises a nucleotide sequence at least 70% (e.g., at least 70%, at least 75%, at least 80%, at least 85%, at least 90%, at least 95%, or at least 99%) identical to the nucleotide sequence of any one of SEQ ID NOs: 10, 12, 52, 54, 56, and 58. In some embodiments, the polynucleotide comprises a nucleotide sequence at least 80% identical to the nucleotide sequence of any one of SEQ ID NOs: 10, 12, 52, 54, 56, and 58. In some embodiments, the polynucleotide comprises a nucleotide sequence of any one of SEQ ID NOs: 10, 12, 52, 54, 56, and 58. Co-Expression of Proteases

In some examples, the method described herein comprising co-expressing the fusion polypeptide comprising the tandem repeats of thaumatin, and optionally the one or more chaperones, with one or more proteases to facilitate processing of the polypeptide comprising the tandem thaumatin repeats, and/or release and secretion of the individual thaumatin proteins.

In some embodiments, the protease is selected from KEX1, KEX2, and Ste13. Proteases of the same yeast strain as the yeast recombinant cell used for expression of the polypeptide may be used. Heterologous proteases from other yeast strains may also be used. As such, in some embodiments, the yeast recombinant cell used in the methods described herein further comprises one or more polynucleotides encoding one or more proteases selected from KEX1, KEX2, and Ste13.

In some examples, the one or more polynucleotides encoding the one or more proteases are provided on one or more vectors (e.g., plasmids). In some examples, the one or more (e.g., 1, 2, 3, or more) polynucleotides encoding the one or more proteases are provided on the same vector as the polynucleotide encoding the fusion polypeptide comprising the tandem thaumatin repeats. In some other examples, the one or more (e.g., 1, 2, 3, 4, 5 or more) polynucleotides encoding the one or more proteases are provided on different vectors as the polynucleotide encoding the polypeptide comprising the tandem thaumatin repeats.

In some examples, the one or more polynucleotides encoding the one or more proteases are integrated into the genome of the recombinant yeast cell. In some cases, the one or more polynucleotides are integrated into a HIS4 or AOX1 locus of the genome of the recombinant yeast cell.

### Host Yeast Strains

Any yeast strain may be suitable as the recombinant yeast cell used in the methods described herein. Non-limiting examples of yeast strains include: Pichia pastoris, Pichia farinose, Pichia anomala, Pichia heedii, Pichia guilliermondii, Pichia kluyveri, Pichia membranifaciens, Pichia norvegensis, Pichia ohmeri, Pichia methanolica, Pichia subpelliculosa, Komagataella phaffii, Komagataella pastoris, Komagataella pseudopastoris, Candida vulgaris, Saccharomyces arboricolus, Saccharomyces bayanus, Saccharomyces bulderi, Saccharomyces cariocanus, Saccharomyces cariocus, Saccharomyces cerevisiae, Saccharomyces cerevisiae var. boulardii, Saccharomyces chevalieri, Saccharomyces dairenensis, Saccharomyces ellipsoideus, Saccharomyces eubayanus, Saccharomyces exiguous, Saccharomyces florentinus, Saccharomyces fragilis, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, and Saccharomyces zonatus. In some embodiments, the recombinant yeast cell in the methods described herein is a recombinant Pichia pastoris cell.

The method may further comprise isolating the thaumatin. In one example, the method comprises isolating thaumatin I. In another example, the method comprises isolating thaumatin II. In some embodiments, the method further comprises isolating both thaumatin I and thaumatin II.

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by GREENE PUBLISHING AND WILEY-INTERSCIENCE, 1987; (the entirety of each of which is hereby incorporated herein by reference).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions. In some embodiments, the yeast cell is of the strain Pichia pastoris.

### Yeast Production Systems

Expression of proteins in eukaryotes is most often carried out in a yeast host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

Moreover, the expression vector typically includes those genetic elements for expression of the recombinant polypeptide in yeast cells. The elements for transcription and translation in the yeast cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

A number of molecular biology techniques have been developed to operably link DNA to vectors via complementary cohesive termini. In one example, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

Alternatively, synthetic linkers containing one or more restriction sites provide are used to operably link the polynucleotide of the subject technology to the expression vector. In some examples, the polynucleotide is generated by restriction endonuclease digestion. In some cases, the nucleic acid molecule is treated with bacteriophage T4 DNA polymerase or E. coli DNA polymerase I, enzymes that remove protruding, 3′-single-stranded termini with their 3′-5′-exonucleolytic activities and fill-in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In some cases, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In some cases, a polynucleotide for incorporation into an expression vector of the subject technology is prepared by the use of PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

Typically, the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS4, GALI, GALIO, ADHI, PGK, PH05, GAPDH, ADCI, TRPI, URA3, LEU2, ENO, TPI (useful for expression in Saccharomyces); AOXI (useful for expression in Pichia).

Termination control regions may also be derived from various genes native to the microbial hosts. A termination site optionally may be included for the microbial hosts described herein.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one example of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As is evident from the foregoing description, certain instances of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### Orally Consumable Products

Some instances of the present disclosure provide compositions comprising the thaumatin produced using the methods described herein. In some cases, the thaumatin produced using the methods described herein can be used, e.g., as sweeteners, in products, e.g., consumable products (e.g., orally consumable products).

In some cases, the consumable products can be, for example, a food product, a beverage product, a nutraceutical, a pharmaceutical, a dietary supplement, a dental hygienic composition, an edible gel composition, a cosmetic product and a tabletop flavoring.

Any one of the consumable products (e.g., orally consumable products) can also have at least one additional sweetener. The at least one additional sweetener can be a natural high intensity sweetener, for example. The additional sweetener can be selected from a stevia extract, a steviol glycoside, stevioside, rebaudioside A, rebaudioside B, rebaudioside C, rebaudioside D, rebaudioside D2, rebaudioside E, rebaudioside F, rebaudioside M, rebaudioside V, rebaudioside W, rebaudioside Z1, rebaudioside Z2, rebaudioside D3, dulcoside A, rubusoside, rebaudioside N, rebaudioside I, rebaudioside G, rebaudioside WB1, rebaudioside WB2, rebaudioside R6-2A, rebaudioside R6-2B, rebaudioside R6-4A, rebaudioside R6-4B, rebaudioside R7-2, steviolbioside, sucrose, high fructose corn syrup, fructose, glucose, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, mogroside IV, siamenoside I, mogroside V, monatin, thaumatin, monellin, L-alanine, glycine, Lo Han Guo, hernandulcin, phyllodulcin, trilobtain, and combinations thereof.

Any one of the consumable products (e.g., orally consumable products) can also have at least one additive. The additive can be, for example, a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

In some instances, the present disclosure provides a beverage product comprising a sweetening amount of thaumatin produced using the methods described herein. Any one of the beverage products can be, for example, a carbonated beverage product and a non-carbonated beverage product. Any one of the beverage products can also be, for example, a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink.

In some instances, any one of the beverage products of the present disclosure can include one or more beverage ingredients such as, for example, acidulants, fruit juices and/or vegetable juices, pulp, etc., flavorings, coloring, preservatives, vitamins, minerals, electrolytes, erythritol, tagatose, glycerine, and carbon dioxide. Such beverage products may be provided in any suitable form, such as a beverage concentrate and a carbonated, ready-to-drink beverage.

In certain instances, any one of the beverage products of the present disclosure can have any of numerous different specific formulations or constitutions. The formulation of a beverage product of the present disclosure can vary to a certain extent, depending upon such factors as the product's intended market segment, its desired nutritional characteristics, flavor profile, and the like. For example, in certain embodiments, it can generally be an option to add further ingredients to the formulation of a particular beverage product. For example, additional (i.e., more and/or other) sweeteners can be added, flavorings, electrolytes, vitamins, fruit juices or other fruit products, tastants, masking agents and the like, flavor enhancers, and/or carbonation typically may be added to any such formulations to vary the taste, mouthfeel, nutritional characteristics, etc.

Exemplary flavorings can be, for example, cola flavoring, citrus flavoring, and spice flavorings. In some examples, carbonation in the form of carbon dioxide can be added for effervescence. In other examples, preservatives can be added, depending upon the other ingredients, production technique, desired shelf life, etc. In certain cases, caffeine can be added. In some cases, the beverage product can be a cola-flavored carbonated beverage, characteristically containing carbonated water, sweetener, kola nut extract and/or other flavoring, caramel coloring, one or more acids, and optionally other ingredients.

As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements can be, for example, nutrients, vitamins, minerals, fiber, fatty acids, herbs, botanicals, amino acids, and metabolites.

As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some cases, nutraceuticals can be used as supplements to food and beverages and as pharmaceutical formulations for enteral or parenteral applications which may be solid formulations, such as capsules or tablets, or liquid formulations, such as solutions or suspensions.

In some cases, dietary supplements and nutraceuticals can further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellifying agents, gel-forming agents, antioxidants and antimicrobials.

As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives. Gels that can be eaten are referred to as “edible gel compositions.” Edible gel compositions typically are eaten as snacks, as desserts, as a part of staple foods, or along with staple foods. Examples of suitable edible gel compositions can be, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids can be, for example, water, dairy fluids, dairy analogue fluids, juices, alcohol, alcoholic beverages, and combinations thereof. Examples of suitable dairy fluids can be, for example, milk, cultured milk, cream, fluid whey, and mixtures thereof. Examples of suitable dairy analogue fluids can be, for example, soy milk and non-dairy coffee whitener.

As used herein, the term “gelling ingredient” refers to any material that can form a colloidal system within a liquid medium. Examples of suitable gelling ingredients can be, for example, gelatin, alginate, carrageenan, gum, pectin, konjac, agar, food acid, rennet, starch, starch derivatives, and combinations thereof. It is well known to those in the art that the amount of gelling ingredient used in an edible gel mix or an edible gel composition can vary considerably depending on a number of factors such as, for example, the particular gelling ingredient used, the particular fluid base used, and the desired properties of the gel.

Gel mixes and gel compositions of the present disclosure can be prepared by any suitable method known in the art. In some embodiments, edible gel mixes and edible gel compositions of the present disclosure can be prepared using other ingredients in addition to the gelling agent. Examples of other suitable ingredients can be, for example, a food acid, a salt of a food acid, a buffering system, a bulking agent, a sequestrant, a cross-linking agent, one or more flavors, one or more colors, and combinations thereof.

Pharmaceutical compositions are also provided comprising thaumatin produced using the methods described herein. In some cases, any one of the pharmaceutical compositions of the present disclosure can be used to formulate pharmaceutical drugs containing one or more active agents that exert a biological effect. Accordingly, in some embodiments, any one of the pharmaceutical compositions of the present disclosure can contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, Pa., USA.

The thaumatin produced using the methods described herein can be used with any suitable dental and oral hygiene compositions known in the art. Examples of suitable dental and oral hygiene compositions can be, for example, toothpastes, tooth polishes, dental floss, mouthwashes, mouth rinses, dentifrices, mouth sprays, mouth refreshers, plaque rinses, dental pain relievers, and the like. Dental and oral hygiene compositions comprising any one of the rebaudiosides provided herein are also provided.

As used herein, “food product composition(s)” refers to any solid or liquid ingestible material that can, but need not, have a nutritional value and be intended for consumption by humans and animals.

Examples of suitable food product compositions can be, for example, confectionary compositions, such as candies, mints, fruit flavored drops, cocoa products, chocolates, and the like; condiments, such as ketchup, mustard, mayonnaise, and the like; chewing gums; cereal compositions; baked goods, such as breads, cakes, pies, cookies, and the like; dairy products, such as milk, cheese, cream, ice cream, sour cream, yogurt, sherbet, and the like; tabletop sweetener compositions; soups; stews; convenience foods; meats, such as ham, bacon, sausages, jerky, and the like; gelatins and gelatin-like products such as jams, jellies, preserves, and the like; fruits; vegetables; egg products; icings; syrups including molasses; snacks; nut meats and nut products; and animal feed.

Food product compositions can also be herbs, spices and seasonings, natural and synthetic flavors, and flavor enhancers, such as monosodium glutamate. In some embodiments, any one of the food product compositions can be, for example, prepared packaged products, such as dietetic sweeteners, liquid sweeteners, granulated flavor mixes, pet foods, livestock feed, tobacco, and materials for baking applications, such as powdered baking mixes for the preparation of breads, cookies, cakes, pancakes, donuts and the like. In other embodiments, any one of the food product compositions can also be diet and low-calorie food and beverages containing little or no sucrose.

### EXAMPLES

### Example 1—Expression of Thaumatin in Pichia pastoris

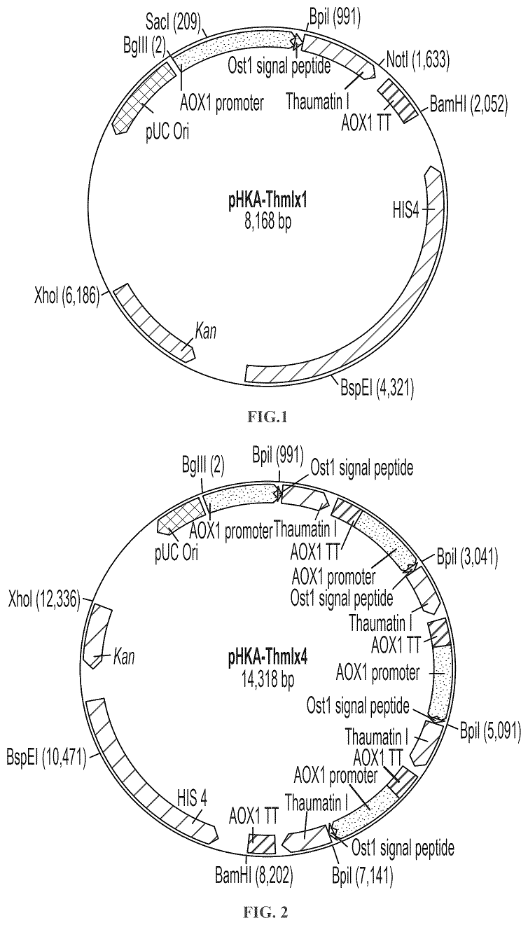

To demonstrate the transformation of Pichia pastoris cells to produce several engineered Pichia strains suitable for secreted thaumatin production, the following experiments were conducted. Full-length DNA fragment of thaumatin I and II genes (SEQ ID NOs: 2 and 4) were codon optimized for Pichia pastoris expression and synthesized for use in the transformation of the Pichia pastoris cells. Thaumatin fragments were inserted in frame after a nucleotide sequence encoding a mating factor signal peptide in pHKA vector (a modified Pichia expression vector) to generate single copy plasmids (pHKA-ThmI and pHKA-ThmII, FIGS. 1 and 2). Multiple signal peptides for the secretion of thaumatin into the culture media were tested. The signal peptide from the S. cerevisiae Dolichyl-diphosphooligosaccharide-protein glycosyltransferase (Ost1) gene (amino acid SEQ ID NO: 5, DNA SEQ ID NO: 6) was determined to be the best for both thaumatin genes. In the plasmid, each expression cassette contains AOX1 promoter, S. cerevisiae Ost1 signal peptide-thaumatin fusion gene and AOX1 transcription terminator. The Ost1 signal peptide-thaumatin fusion protein can be cleaved by endogenous signal peptidase and release mature thaumatin peptides (SEQ ID NO: 1 and 3) into extracellular space.

To generate the multiple copies of the expression cassette in vitro, the above plasmid was digested with BspEI and BglII or BspEI and BamHI. The fragments containing the thaumatin coding sequence were gel-purified then ligated together. Resulting E. coli colonies were screened by digestion with BglII and BamHI to find colonies with an insert that is double the size of the signal expression cassette, which are plasmids containing 2 expression cassettes. This procedure was repeated on the 2 copies plasmid to generate pHKA Pichia expression plasmids harboring 4 copies of identical thaumatin expression cassettes (pHKA-ThmIx4 and pHKA-ThmIIx4, FIGS. 2 and 4).

Identified plasmids were linearized at the HIS4 gene with a BspEI digestion. The linearized expression plasmid was transformed into Pichia pastoris (GS115) cells using known methods and the expression cassette was integrated into the His 4 locus of Pichia genome. After screening, the positive strains were identified, as summarized in Table 1.

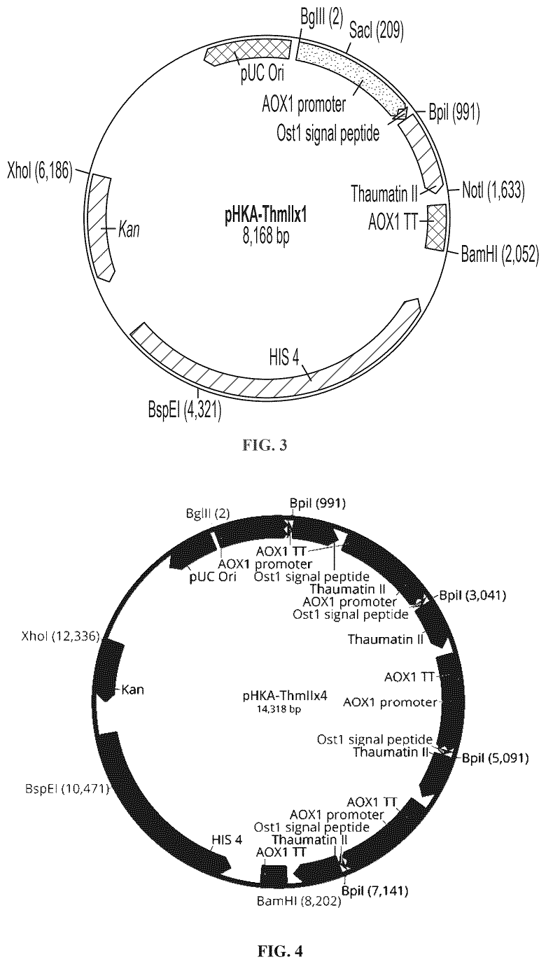

TABLE 1
Summary of Pichia pastoris strains

| | | | Thaumatin | Thaumatin | | |
|---|---|---|---|---|---|---|
| Strain | Plasmid | Plasmid | amino acid | DNA | Description | Integration |
| name | name | map | sequence | sequence | of plasmid | locus |
| ThmIx1 | pHKA- | FIG. 1 | SEQ ID | SEQ ID | 1 thaumatin | His 4 |
| | ThmIx1 | | NO: 1 | NO: 2 | I expression | |
| | | | | | cassette | |
| ThmIx4 | pHKA- | FIG. 2 | | | 4 thaumatin | His 4 |
| | ThmIx4 | | | | I expression | |
| | | | | | cassettes | |
| ThmIIx1 | pHKA- | FIG. 3 | SEQ ID | SEQ ID | 1 thaumatin | His 4 |
| | ThmIIx1 | | NO: 3 | NO: 4 | II expression | |
| | | | | | cassette | |
| ThmIIx4 | pHKA- | FIG. 4 | | | 4 thaumatin | His 4 |
| | ThmIIx4 | | | | II expression | |
| | | | | | cassettes | |

To demonstrate thaumatin production, the following experiment was conducted. Single colonies of the Pichia pastoris strains were inoculated in BMGY medium in a 24 wells plate or baffled flask and grown at 28-30 °C in a shaking incubator (250-300 rpm) until the culture reached an OD₆₀₀ of 2-6 (log-phase growth). The cells were harvested by centrifuging and resuspended to an OD₆₀₀ of 1.0 in BMM/BMMY medium to induce expression. 100% methanol was added to the BMMY medium to a final concentration of 1% methanol every 24 hours to maintain induction of expression. The medium was harvested at different induction time by centrifugation and analyzed by SDS-PAGE, HPLC and LC MS as described below.

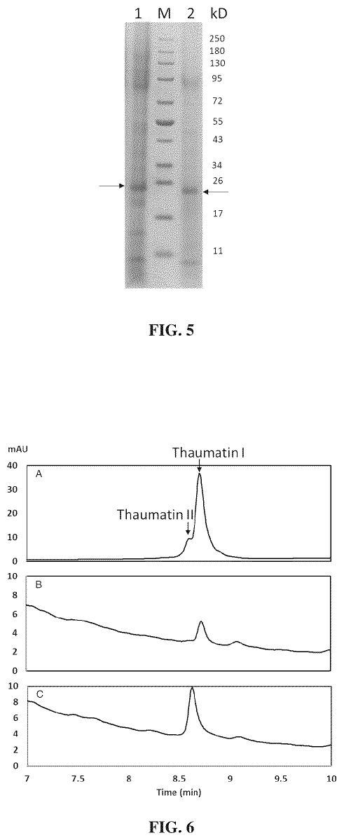

In order to identify thaumatin production, multiple methods were used to detect thaumatin in the products. Medium samples were subjected to electrophoresis on a 10-20% SDS-PAGE gel. As shown in FIG. 5, there were 22 kDa bands in all thaumatin expressing media samples, indicating thaumatin production in engineered Pichia strain. Increasing the number of thaumatin expression cassette copies through in vitro multimerization can increase thaumatin production in Pichia (FIG. 5). In order to confirm that Pichia-produced thaumatin is correctly folded with all native disulfide bonds, these samples were analyzed by HPLC and LC MS analysis as compared to thaumatin standard.

HPLC analysis was performed using a C4 HPLC column. A linear gradient increased from 20%-40% 0.1% TFA in water: 0.1% TFA in acetonitrile over 10 minutes then dropped down to 20% for an additional 5 minutes at a flow rate of 0.6 mL/min. Thaumatin I standard elutes at 8.7 minutes and thaumatin II standard elutes at 8.6 minutes. When the media samples of ThmI and ThmII strains were precipitated with ammonium sulfate and resuspended in water, a peak with a same retention time to the standard was observed (FIG. 6).

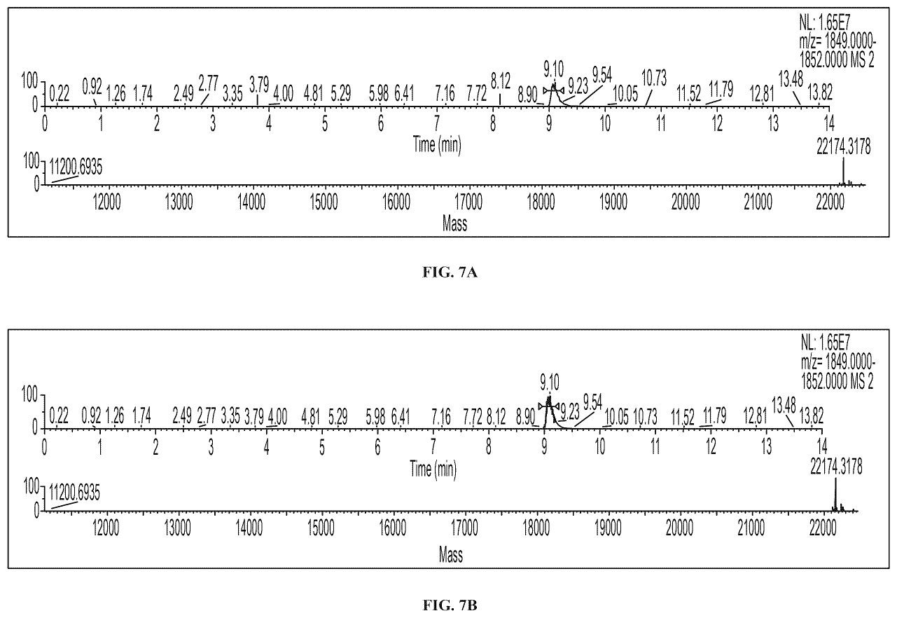

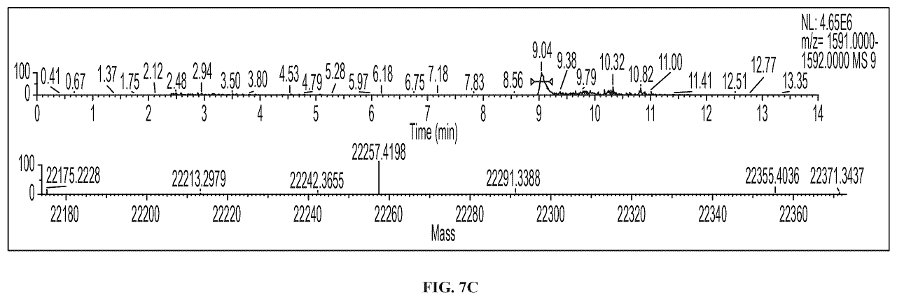

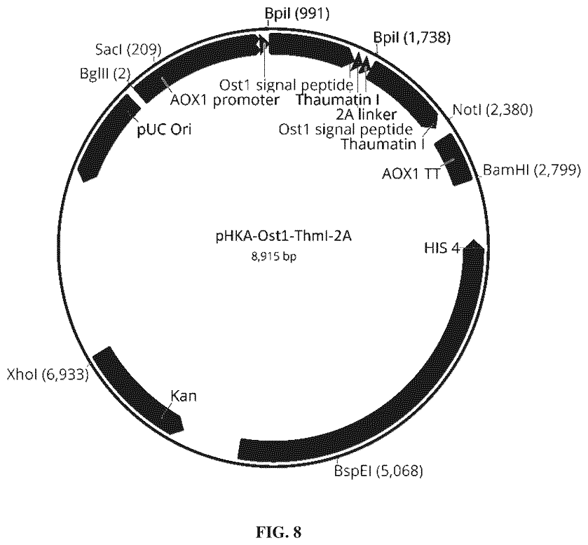

Samples were analyzed by LC-MS using a C4 column. Mobile phase A was 0.1% formic acid in water, and mobile phase B was 0.1% formic acid in acetonitrile. The flow rate was set at 0.2 ml/minute. Mass spectrometry analysis of the samples was done on the Q Exactive Hybrid Quadrupole-Orbitrap Mass Spectrometer (Thermo Fisher Scientific) with an optimized method in positive ion mode. The produced peptides in ThmI and ThmII strains have similar retention times as a thaumatin standard purchased from Sigma-Aldrich. The standard is a mixture of type I and type II thaumatin extracted from the Thaumatococcus daniellii plant. The produced thaumatin I peptide has same mass ([M+H]⁺: 22174 m/z) as the major compound in the thaumatin standard (thaumatin I with eight disulfide bonds). The Pichia produced thaumatin II had a mass of 22257 m/z, matching that of the expected mass of the compound with the eight disulfide bonds. These results provided the evidence supporting thaumatin production in engineered Pichia strain (ThmI, ThmII etc.) (FIGS. 7A-7C).

### Example 2: Expression of Thaumatin I and II Tandem Repeats Using a 2A Linker

Another expression strategy for thaumatin involves the use of 2A peptide linkers between signal peptide-thaumatin open reading frames. The 2A linker pauses translation of mRNA to protein at the N-terminal glycine residue, then translation is re-initiated at the proline residue at the C-terminus of the 2A peptide. Of the various sequences of known 2A linkers, we selected the Equine rhinitis B virus (ERBV-1) 2A linker (amino acid SEQ ID NO: 7 and DNA SEQ ID NO: 8) one that has shown to be highly efficient in pausing and restarting translation in yeast similar to P. pastoris.

The 2A linker was added to the pHKA-Ost1-ThmI and ThmII plasmids by amplifying the thaumatin gene with overlaps that code for the 2A linker with Gibson assembly. The assembly was transformed into 10G colonies and the correct sequence was confirmed with Sanger sequencing. Correct plasmids were linearized by BspEI digestion and transformed into Pichia cell.

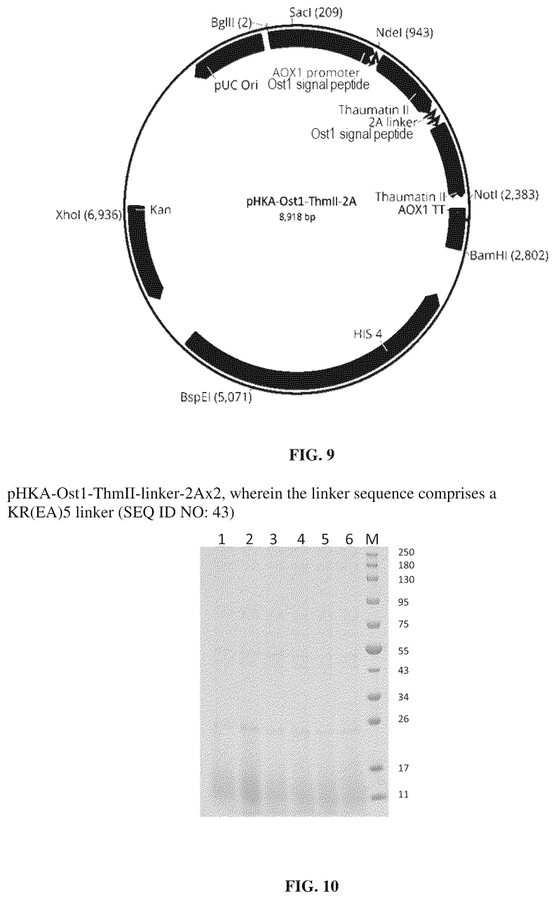

TABLE 2
Summary of strains and plasmids (2A linker)

| | | Fusion protein | Fusion | | |
|---|---|---|---|---|---|
| | | Amino acid | gene DNA | Plasmid | Integration |
| Strain name | Plasmid name | sequence | sequence | map | locus |
| ThmI-2A2 | pHKA-Ost1-ThmI-2A | SEQ ID | SEQ ID | FIG. 8 | HIS4 |
| | | NO: 9 | NO: 10 | | |
| ThmII-2A2 | pHKA-Ost1-ThmII-2A | SEQ ID | SEQ ID | FIG. 9 | HIS4 |
| | | NO: 11 | NO: 12 | | |

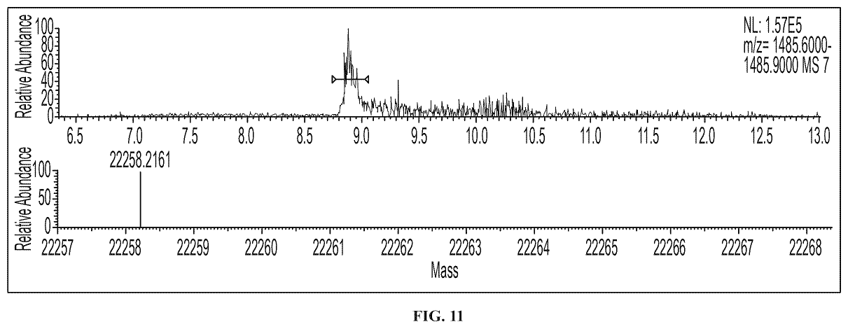

To demonstrate thaumatin I and II production, the following experiment was conducted. Single colonies of the Pichia pastoris strains were inoculated in BMGY medium in a 24 well plate or baffled flask and grown at 28-30 °C in a shaking incubator (250-300 rpm) until the culture reached an OD₆₀₀ of 2-6 (log-phase growth). The cells were harvested by centrifuging and resuspended to an OD₆₀₀ of 1.0 in BMM/BMMY medium to induce expression. Methanol 100% was added to the BMMY medium to a final concentration of 1% methanol every 24 hours to maintain induction of expression. The medium was harvested at different induction time by centrifugation and subjected to SDS-PAGE, HPLC and LC MS analysis as described below (FIGS. 10 and 11).

ThmII-2A2 strain can produce thaumatin II protein. The produced thaumatin II can be detected by SDS-PAGE (FIG. 10) and LC MS (FIG. 11).

### Example 3—Improvement Thaumatin Folding, Secretion and Production in Pichia pastoris

For the thaumatin peptide to have its characteristic sweetness, the six disulfide bonds must be formed in the correct positions. While P. pastoris is a good host for disulfide bond formation, the overexpression of a heterologous disulfide bonded product can overwhelm the cell's native capacity. To improve the amount of correctly folded thaumatin produced by above identified Pichia strains, a series of chaperone and proteins related to protein expression, secretion, folding and disulfide formation were over expressed in thaumatin production Pichia strains. These chaperones and proteins were selected from P. pastoris or plant to be heterologous expressed with thaumatin in Pichia. While none of the chaperones involved in disulfide bond formation in Thaumatococcus daniellii have been identified, the disulfide bond formation system of the closely related Arabidopsis thaliana has been characterized (Table 3).

Protein disulfide isomerase (PDI) is a chaperone localized primarily in the endoplasmic reticulum that aids in forming disulfide bonds between cysteine residues. Overexpressing PDI in P. pastoris has also been shown to improve the expression of certain non-disulfide bond containing proteins. ER oxidoreductin (ERO) proteins work in tandem with PDI by donating oxidating equivalents for disulfide bond formation. ERVs are a family of sulfhydryl oxidases that play a similar role as EROs but may also directly catalyze disulfide bond formation. HAC1 is a transcriptional regulator of the unfolded protein response in P. pastoris. GPX1 is a cytosolic peroxidase that is involved in cellular redox balancing. KAR2 codes for the ER chaperone BiP that aids in proper folding and directs misfolded proteins to be degraded. The genes SLY1 and SEC1 regulate vesicle traffic from the ER to the Golgi and from the Golgi to the extracellular membrane respectively. All selected transcription regulator, chaperones and disulfide bond formation related proteins were list in Table 3.

TABLE 3
Selection of candidates for co-expression
in thaumatin production strain

| | GenBank | Amino acid | |
|---|---|---|---|
| Name | accession number | sequence | DNA sequence |
| PpPDI1 | CAC33587.1 | SEQ ID NO: 13 | SEQ ID NO: 14 |
| AtPDI1 | Q8VX13.1 | SEQ ID NO: 15 | SEQ ID NO: 16 |
| HAC1 | 254565857 | SEQ ID NO: 17 | SEQ ID NO: 18 |
| AtERO1 | NP_177372.1 | SEQ ID NO: 19 | SEQ ID NO: 20 |
| AtERO2 | NP_181430.2 | SEQ ID NO: 21 | SEQ ID NO: 22 |
| AtERV1 | XM_002492510.1 | SEQ ID NO: 23 | SEQ ID NO: 24 |
| PpERV2 | ANZ73509.1 | SEQ ID NO: 25 | SEQ ID NO: 26 |
| PpERO1 | ANZ74048.1 | SEQ ID NO: 27 | SEQ ID NO: 28 |
| PpKAR2 | ANZ77450.1 | SEQ ID NO: 29 | SEQ ID NO: 30 |
| PpSEC1 | CAY67514.1 | SEQ ID NO: 31 | SEQ ID NO: 32 |
| PpSLY1 | CCA40908.1 | SEQ ID NO: 33 | SEQ ID NO: 34 |
| PpGPX1 | BAH57503.1 | SEQ ID NO: 35 | SEQ ID NO: 36 |

Chaperone genes were cloned into a modified pPICZ vector that has an NdeI site after the AOX1 promoter in place of the EcoRI site. To construct vectors with multiple chaperones or multiple copies of the same chaperone, the vector containing chaperone to be added was digested with BglII and BamHI to release the expression cassette. The expression cassette was then ligated into a second chaperone vector linearized with BamHI. A BglII and BamHI digest was performed on the resulting plasmids to confirm the insertion of the chaperone in the proper orientation. Generated vectors were linearized at the AOX1 promoter with SacI restriction enzyme and used to transform GS115 P. pastoris with thaumatin expression cassettes integrated at the HIS4 locus. Colonies with chaperone integration were selected on YPD-Zeocin and confirmed by colony PCR.

As the selected chaperones often work in tandem with other enzymes to improve secretion or disulfide bonding, expression vectors with multiple genes were generated. To ensure integration at the AOX1 locus, the promoter from the added genes needed to be replaced. The GAP1 and CAT1 promoters were amplified from GS115 genomic DNA with primers that added BglII and NdeI sites to the 5′ and 3′ ends respectively. The AOX1 promoter was then excised from the pPICZ vectors with a BglII/NdeI digest and replaced with the GAP1 and CAT1 promoters. Various combinations of different expression cassettes were generated by ligating a chaperone with GAP1 or CAT1 promoter excised from its expression vector with a BamHI/BglII digest into a pPICZ AOX1 chaperone vector linearized with BamHI. All plasmids are listed below in Table 4.

TABLE 4
Summary of selected plasmids of single and combination
of different chaperones for co-expression

| Name | Chaperone Gene | Promoter | Parent strain |
|---|---|---|---|
| pPICZ-PpPDI1 | PpPDI1 (SEQ ID NO: 14) | AOX1 | Thm II |
| pPICZ-AtPDI1 | AtPDI1 (SEQ ID NO: 16) | AOX1 | Thm II |
| pPICZ-HAC1 | HAC1 (SEQ ID NO: 18) | AOX1 | Thm II |
| pPICZ- | PpPDI1 (SEQ ID NO: 14) | AOX1 | Thm II |
| PpPDI1/PpKAR1 | PpKAR2 (SEQ ID NO: 30) | CAT1 | |
| pPICZ- | PpPDII (SEQ ID NO: 14) | AOX1 | Thm II |
| PpPDI1/PpERO1/ | PpERO1 (SEQ ID NO: 28) | GAP1 | |
| PpERV2 | PpERV2 (SEQ ID NO: 26) | CAT1 | |
| pPICZ- | AtPDI1 (SEQ ID NO: 16) | AOX1 | Thm II |
| AtPDI1/AtERV1/ | AtERV1(SEQ ID NO: 24) | CAT1 | |
| AtERO1 | AtERO1 (SEQ ID NO: 20) | CAT1 | |

To demonstrate improvement of thaumatin production with chaperone co-expression, confirmed colonies were grown overnight in BMGY media in 24 well plates. The next day the cells were resuspended in 2 mL BMMY media to an OD₆₀₀ of 1.0. The cultures were induced at 30 °C for 48 hours with the additional feeding of 1% methanol to each well twice daily. The cells were harvested and spun down. The supernatant was analyzed by SDS-PAGE, HPLC and LC MS.

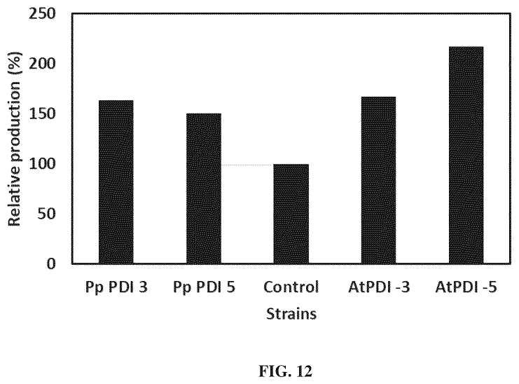

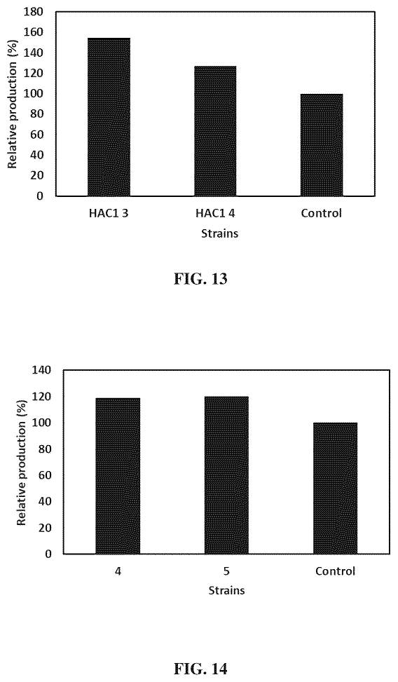

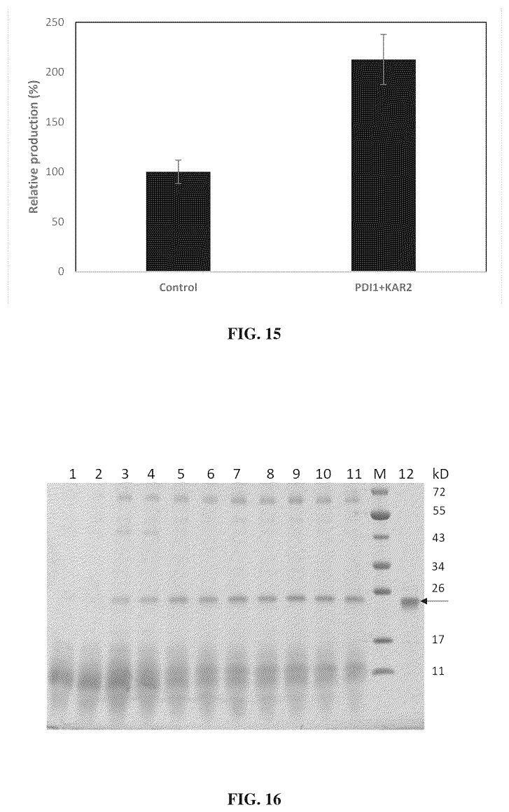

Co-expression of PpPDI1 or AtPDI1 can increase thaumatin production. As shown in FIG. 12, co-expression strains had higher thaumatin production than the parent strain. Co-expression of HAC1 transcription regulator also can increase thaumatin production (FIG. 13). Co-expression of multiple chaperons and disulfide bond formation related proteins (PDI, ERO and ERV), either from Pichia pastoris and Arabidopsis, can increase thaumatin production in Thm II strain (FIG. 14). Combining the disulfide bond formation chaperone PDI1 and the ER membrane trafficking chaperone PpKAR2 also increases the amount of thaumatin secreted by Pichia (FIG. 15).

### Example 5: Fermentation, Purification and Identification of Thaumatin I and Thaumatin II

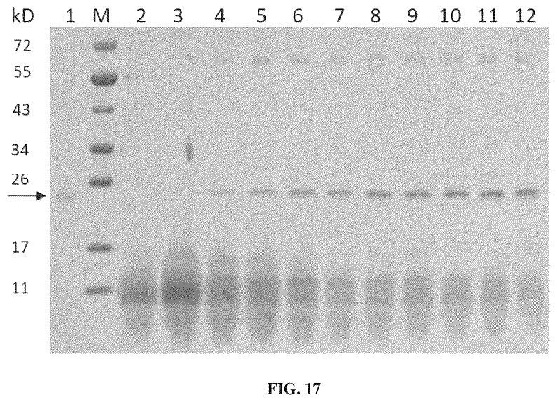

Identified thaumatin I and II production strains (ThmIx4 and ThmIIx4) were cultured in 3 L fermenters for thaumatin production. Seed cultures were inoculated into rich media with glycerol and methanol was continually fed into the medium for thaumatin induction after glycerol was fully consumed. Medium samples were collected at different time points and analyzed by SDS-PAGE and HPLC. As shown in FIG. 16, no thaumatin I was detected before methanol feeding (FIG. 16: 1 and 2); After methanol feeding, thaumatin I production increased along with the induction time throughout the 138 hr fermentation time (FIG. 16: 3-11). As shown in FIG. 17, no thaumatin II was detected before methanol feeding (FIG. 17: 2 and 3); After methanol feeding, thaumatin II production increased along with the induction time throughout the 138 hr fermentation time (FIG. 17: 4-12).

### Example 6—Alternate 2A Peptide with Alpha Mating Factor Spacer Peptides

An alternate method of expressing thaumatin in a tandem repeat was demonstrated by combining the 2A polypeptide strategy with a linker that mimics the alpha mating factor. The mating factor mimic linker can be cleaved by endogenous P. pastoris proteases to improve the release of thaumatin monomers from tandem repeats. The length of mating factor linkers varies between yeast species. Our experimentation has shown linkers with five repeats of the EA dipeptide are efficiently cleaved by endogenous P. pastoris KEX2 protease. (amino acid SEQ ID NO: 37 and DNA SEQ ID NO: 38) has shown to be highly efficient in pausing and restarting translation in yeast similar to P. pastoris. When combined with the 2A peptide, the yeast cell will process the tandem repeat polypeptide to thaumatin by starting and stopping translation at each 2A peptide. Extra C-terminal amino acids will be cleaved by the combined action of the KEX2 and KEX1 proteases.

The mating factor linker was added to the pHKA-Ost1-ThmII-2A plasmid by amplifying the end of the first thaumatin tandem repeat in the reverse direction and the 2A peptide in the forward direction with overlaps that code for the mating factor linker DNA sequence. The DNA fragments were assembled by Gibson assembly and sequence confirmed. The PCR and DNA assembly was repeated to generate plasmids with 8 thaumatin sequences separated by 7 linkers. Alternatively, the pHKA-Ost1-ThmII-linker-2Ax2, wherein the linker sequence comprises a KR(EA)5 linker (SEQ ID NO: 43), plasmid was multimerized by the methods described in example 1 until a plasmid with 8 copies of the Ost1-ThmII-linker-2Ax2, wherein the linker sequence comprises a KR(EA)5 linker (SEQ ID NO: 43), coding sequence was obtained. Plasmids were linearized by BspEI digestion and transformed into Pichia cells by electroporation. The strains secrete thaumatin II of the correct mass on both SDS-PAGE (FIG. 24) and LCMS. The plasmids and strains generated for this group of strains are listed in Table 5.

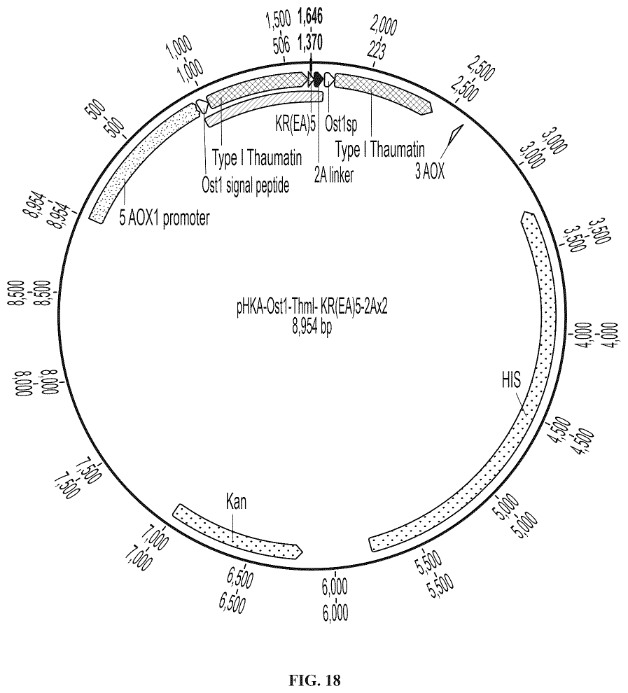

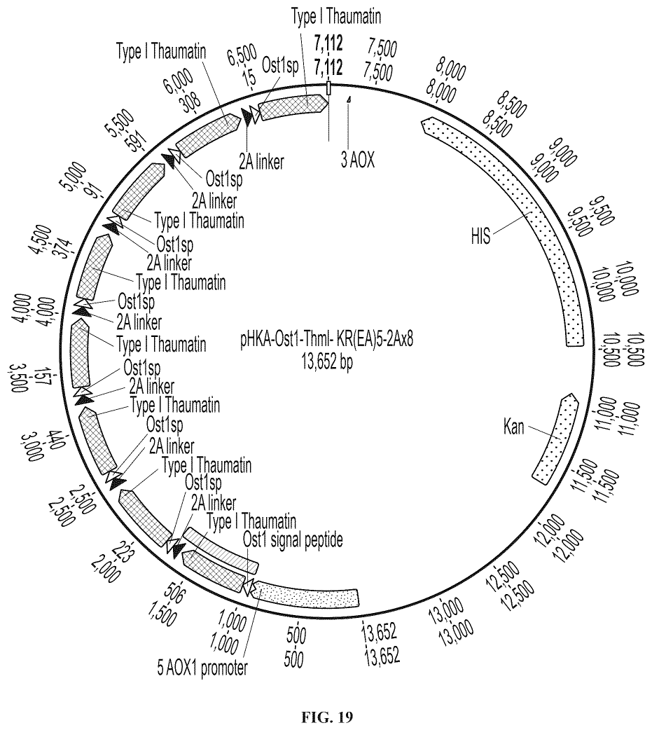

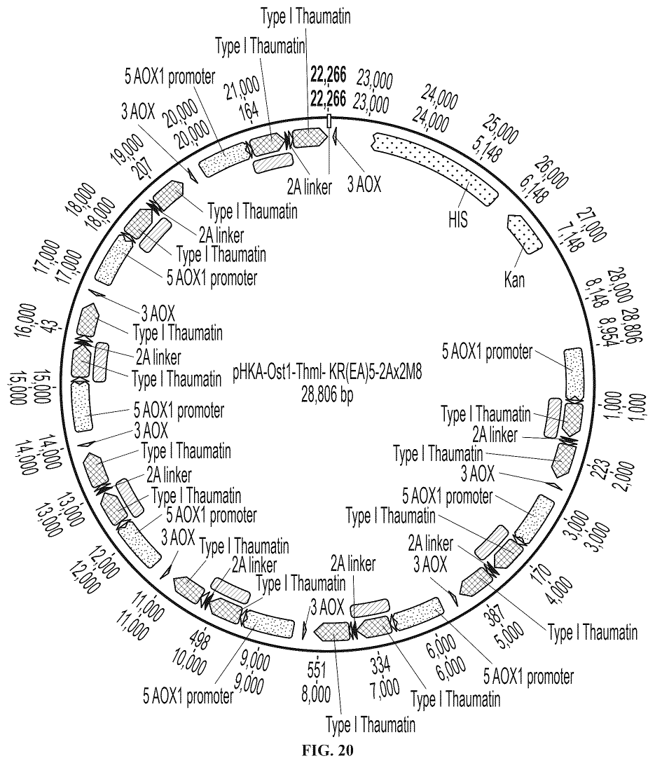

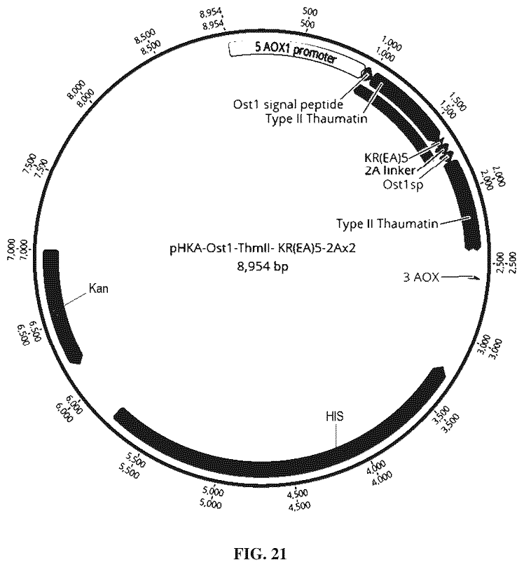

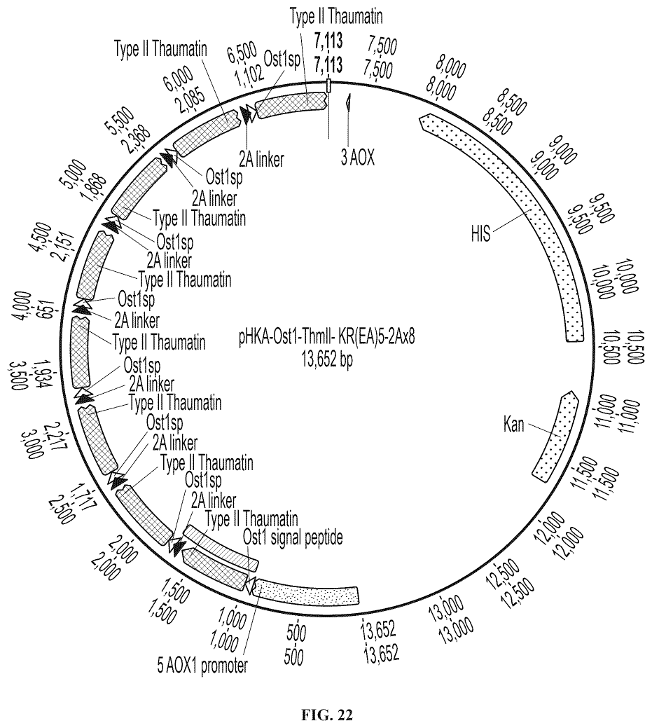

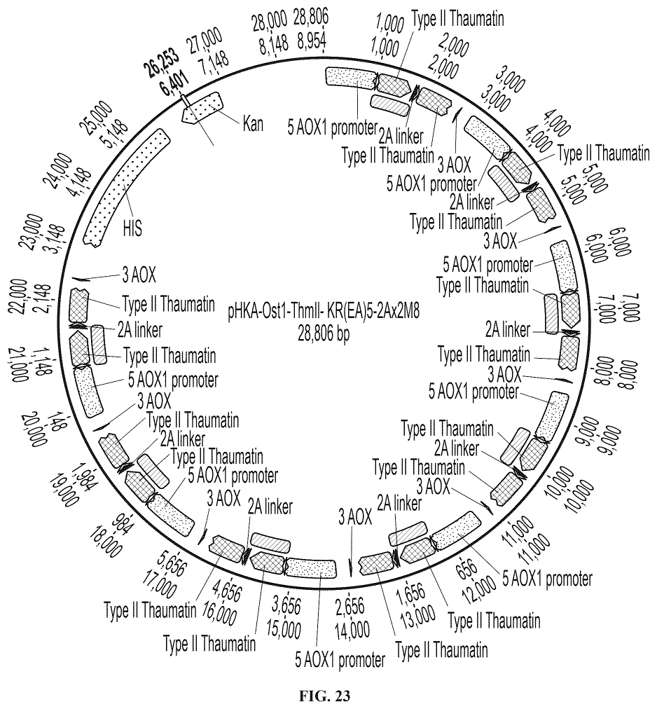

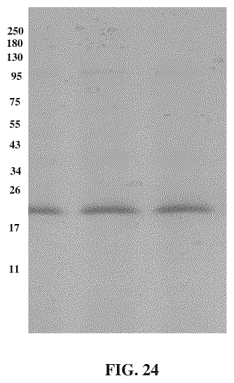

TABLE 5
Summary of Linker-2A, wherein the linker sequence comprises
a KR(EA)5 linker (SEQ ID NO: 43), plasmids.

| | | Fusion protein | Fusion | | |
|---|---|---|---|---|---|
| | | Amino acid | gene DNA | Plasmid | Integration |
| Strain name | Plasmid name | sequence | sequence | map | locus |
| ThmI- linker- | pHKA-Ost1- | SEQ ID | SEQ ID | FIG. 18 | HIS4 |
| 2Ax2, wherein | ThmI- linker- | NO: 51 | NO: 52 | | |
| the linker | 2Ax2, wherein | | | | |
| sequence | the linker | | | | |
| comprises a | sequence | | | | |
| KR(EA)5 linker | comprises a | | | | |
| (SEQ ID NO: 43). | KR(EA)5 linker | | | | |
| | (SEQ ID NO: 43). | | | | |
| ThmI- linker- | pHKA-Ost1- | SEQ ID | SEQ ID | FIG. 19 | HIS4 |
| 2Ax8, wherein | ThmI- linker- | NO: 53 | NO: 54 | | |
| the linker | 2Ax8, wherein | | | | |
| sequence | the linker | | | | |
| comprises a | sequence | | | | |
| KR(EA)5 linker | comprises a | | | | |
| (SEQ ID NO: 43). | KR(EA)5 linker | | | | |
| | (SEQ ID NO: 43). | | | | |
| ThmI- linker- | pHKA-Ost1- | SEQ ID | SEQ ID | FIG. 20 | HIS4 |
| 2Ax2M8, | ThmI- linker- | NO: 51 | NO: 52 | | |
| wherein the | 2Ax2M8, wherein | | | | |
| linker sequence | the linker | | | | |
| comprises a | sequence | | | | |
| KR(EA)5 linker | comprises a | | | | |
| (SEQ ID NO: 43). | KR(EA)5 linker | | | | |
| | (SEQ ID NO: 43). | | | | |
| ThmII- linker- | pHKA-Ost1- | SEQ ID | SEQ ID | FIG. 21 | HIS4 |
| 2Ax2, wherein | ThmII- linker- | NO: 55 | NO: 56 | | |
| the linker | 2Ax2, wherein | | | | |
| sequence | the linker | | | | |
| comprises a | sequence | | | | |
| KR(EA)5 linker | comprises a | | | | |
| (SEQ ID NO: 43). | KR(EA)5 linker | | | | |
| | (SEQ ID NO: 43). | | | | |
| ThmII- linker- | pHKA-Ost1- | SEQ ID | SEQ ID | FIG. 22 | HIS4 |
| 2Ax8, wherein | ThmII- linker- | NO: 57 | NO: 58 | | |
| the linker | 2Ax8, wherein | | | | |
| sequence | the linker | | | | |
| comprises a | sequence | | | | |
| KR(EA)5 linker | comprises a | | | | |
| (SEQ ID NO: 43). | KR(EA)5 linker | | | | |
| | (SEQ ID NO: 43). | | | | |
| ThmII- linker- | pHKA-Ost1- | SEQ ID | SEQ ID | FIG. 23 | HIS4 |
| 2Ax2M8, | ThmII- linker- | NO: 55 | NO: 56 | | |
| wherein the | 2Ax2M8, wherein | | | | |
| linker sequence | the linker | | | | |
| comprises a | sequence | | | | |
| KR(EA)5 linker | comprises a | | | | |
| (SEQ ID NO: 43). | KR(EA)5 linker | | | | |
| | (SEQ ID NO: 43). | | | | |

### REFERENCES

1. Van der Wel H., Loeve K. (1972). Isolation and characterization of thaumatin I and II, the sweet-tasting proteins from Thaumatococcus daniellii Benth. Eur. J. Biochem. 31 221-225.
2. Ide N., Kaneko R., Wada R., Mehta A., Tamaki S., Tsuruta T. (2007a). Cloning of the thaumatin I cDNA and characterization of recombinant thaumatin I secreted by Pichia pastoris. Biotechnol. Prog. 23 1023-1030.
3. Joseph J A, Akkermans S, Nimmegeers P. Van Impe J F M. (2019). Bioproduction of the Recombinant Sweet Protein Thaumatin: Current State of the Art and Perspectives. Front Microbiol. 8; 10:695.

1. A method of producing thaumatin, the method comprising culturing a recombinant yeast cell comprising a polynucleotide encoding a fusion polypeptide comprising tandem repeats of thaumatin.

2. The method of claim 1, wherein each thaumatin in the tandem repeat of thaumatin:
(i) comprises the amino acid sequence of SEQ ID NO: 1;
(ii) comprises the amino acid sequence of SEQ ID NO: 3; and/or
(iii) is separated by a spacer.

3.-4. (canceled)

5. The method of claim 2, wherein the spacer:
(i) comprises a protease cleavage site for a yeast protease;
(ii) comprises the amino acid sequence of any one of SEQ ID NOs: 39, 41, and 43; and/or
(iii) comprises a 2A linker, optionally wherein the 2A linker comprises the amino acid sequence of SEQ ID NO: 7.

6. (canceled)

7. The method of claim 1, wherein the fusion polypeptide:
(i) comprises 2-8 repeats of thaumatin;
(ii) comprises an amino acid sequence at least 80% identical to the amino acid sequence of any one of SEQ ID NOs: 9, 11, 51, 53, 55 and 57;
(iii) comprises the amino acid sequence of any one of SEQ ID NOs: 9, 11, 51, 53, 55 and 57; and/or
(iv) further comprises an N-terminal signal peptide.

8.-10. (canceled)

11. The method of claim 7, wherein the signal peptide:
(i) is a yeast alpha mating factor signal peptide, optionally wherein the yeast alpha mating factor signal peptide comprises the amino acid sequence of SEQ ID NO: 37; and/or
(ii) is an Ost1 signal peptide, wherein the Ost1 signal peptide comprises the amino acid sequence of SEQ ID NO: 5.

12.-13. (canceled)

14. The method of claim 1, wherein the polynucleotide:
(i) is operably linked to a promoter, optionally wherein the promoter is an AOX1 promoter;
(ii) is operably linked to a transcription terminator, optionally wherein the transcription terminator is an AOX1 terminator;
(iii) polynucleotide is provided on a vector, optionally wherein the vector is a plasmid; and/or
(iv) is integrated into the genome of the recombinant yeast cell, optionally wherein the polynucleotide is integrated into a HIS4 locus of the genome of the recombinant yeast cell.

15.-17. (canceled)

18. The method of claim 1, wherein the recombinant yeast cell further comprises one or more polynucleotides encoding one or more chaperones selected from: PDI1, HAC1, ERO1, ERO2, ERV1, ERV2, KAR2, SEC1, SLY1, and GPX1, optionally wherein the recombinant yeast cell further comprises a polynucleotide encoding PpPDI1, a polynucleotide encoding HAC1, one or more polynucleotides encoding PpPDI1, PpERO1, and PpERV2, or one or more polynucleotides encoding AtPDI1, AtERV1, and AtERO1.

19. The method of claim 18, wherein the one or more polynucleotides encoding the one or more chaperones:
(i) are provided on one or more vectors; and/or
(ii) are integrated into the genome of the recombinant yeast cell, optionally wherein the one or more polynucleotides are integrated into a HIS4 locus of the genome of the recombinant yeast cell.

20. (canceled)

21. The method of claim 1, wherein the recombinant yeast cell further comprises one or more polynucleotides encoding one or more proteases selected from KEX1, KEX2, and Ste13.

22. The method of claim 21, wherein the one or more polynucleotides encoding the one or more proteases:
(i) are provided on one or more vectors; and/or
(ii) are integrated into the genome of the recombinant yeast cell, optionally wherein the one or more polynucleotides are integrated into a AOX1 locus of the genome of the recombinant yeast cell.

23. (canceled)

24. The method of claim 1, wherein the yeast cell is a Pichia pastoris cell.

25. The method of claim 1, further comprising isolating the thaumatin.

26. The method of claim 25, wherein the isolated thaumatin is selected from the group consisting of thaumatin I and thaumatin II.

27. Thaumatin produced using the method of claim 1, for use as a sweetener.

28. A composition or a consumable product comprising the thaumatin produced using the method of claim 1.

29. (canceled)

30. The composition or the consumable product of claim 28, further comprising:
(i) a second sweetener;
(ii) a rebaudioside; and/or
(iii) at least one additive is selected from the group consisting of a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

31.-32. (canceled)

33. The consumable product of claim 28, wherein the consumable product is selected from: a food product, a beverage product, a nutraceutical, a pharmaceutical, a dietary supplement, a dental hygienic composition, an edible gel composition, a cosmetic product and a tabletop flavoring.

34. The consumable product of claim 33, wherein the beverage product is:
(i) selected from the group consisting of a carbonated beverage product and a non-carbonated beverage product; and/or
(ii) selected from the group consisting of a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink.

35.-36. (canceled)

37. A polypeptide comprising an amino acid sequence that is at least 80% identical to any one of SEQ ID NOs: 9, 11, 51, 53, 55 and 57.

38.-51. (canceled)

52. A polynucleotide comprising, a nucleotide sequence that is at least 80% identical to any one of SEQ ID NOs: 10, 12, 52, 54, 56, and 58.

53. (canceled)


SEQUENCE LISTING (58 sequences)

SEQ ID NO: 1 - protein, 207 aa, unidentified
  source 1..207: note = Pichia pastoris
ATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 2 - DNA, 621 bp, unidentified
  source 1..621: mol_type = genomic DNA; note = Pichia pastoris
gctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgct

SEQ ID NO: 3 - protein, 207 aa, unidentified
  source 1..207: note = Pichia pastoris
ATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 4 - DNA, 621 bp, unidentified
  source 1..621: mol_type = genomic DNA; note = Pichia pastoris
gctactttcgaaattgttaacagatgttcttacactgtttgggctgctgcttctaagggtgacgctgctttggatgctggtggtagacaattgaactctggtgaatcttggactattaatgttgaaccaggtactaagggtggtaaaatttgggctagaactgattgttactttgatgattctggtagaggtatttgtagaactggtgactgtggtggtttgttgcaatgtaagagatttggtagaccaccaactactttggctgaattttctttgaaccaatatggtaaagactacattgatatctctaacattaagggtttcaacgttccaatggatttctctcctactactagaggttgtagaggtgttagatgtgctgctgatattgttggtcaatgtccagctaaattgaaggctcctggtggtggttgtaatgatgcttgtactgtttttcaaacttctgaatactgttgtactactggtaaatgtggtcctactgaatattctagattcttcaaaagactgtgtcctgatgctttttcttacgttttggataagcctactactgttacttgtcctggttcttctaactacagagttactttttgtcctactgct

SEQ ID NO: 5 - protein, 22 aa, synthetic construct
MRQVWFSWIVGLFLCFFNVSSA

SEQ ID NO: 6 - DNA, 66 bp, synthetic construct
  source 1..66: mol_type = other DNA
atgaggcaggtttggttctcttggattgtgggattgttcctatgttttttcaacgtgtcttctgct

SEQ ID NO: 7 - protein, 20 aa, synthetic construct
GATNFSLLKLAGDVELNPGP

SEQ ID NO: 8 - DNA, 60 bp, synthetic construct
  source 1..60: mol_type = other DNA
ggagcaactaatttcagtctcctgaagttggccggtgacgttgaactgaaccctggaccg

SEQ ID NO: 9 - protein, 478 aa, synthetic construct
MRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 10 - DNA, 1434 bp, synthetic construct
  source 1..1434: mol_type = other DNA
atgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgct

SEQ ID NO: 11 - protein, 478 aa, synthetic construct
MRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 12 - DNA, 1434 bp, synthetic construct
  source 1..1434: mol_type = other DNA
atgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttttgtcctactgctggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgct

SEQ ID NO: 13 - protein, 517 aa, unidentified
  source 1..517: note = Pichia pastoris
MQFNWDIKTVASILSALTLAQASDQEAIAPEDSHVVKLTEATFESFITSNPHVLAEFFAPWCGHCKKLGPELVSAAEILKDNEQVKIAQIDCTEEKELCQGYEIKGYPTLKVFHGEVEVPSDYQGQRQSQSIVSYMLKQSLPPVSEINATKDLDDTIAEAKEPVIVQVLPEDASNLESNTTFYGVAGTLREKFTFVSTKSTDYAKKYTSDSTPAYLLVRPGEEPSVYSGEELDETHLVHWIDIESKPLFGDIDGSTFKSYAEANIPLAYYFYENEEQRAAAADIIKPFAKEQRGKINFVGLDAVKFGKHAKNLNMDEEKLPLFVIHDLVSNKKFGVPQDQELTNKDVTELIEKFIAGEAEPIVKSEPIPEIQEEKVFKLVGKAHDEVVFDESKDVLVKYYAPWCGHCKRMAPAYEELATLYANDEDASSKVVIAKLDHTLNDVDNVDIQGYPTLILYPAGDKSNPQLYDGSRDLESLAEFVKERGTHKVDALALRPVEEEKEAEEEAESEADAHDEL

SEQ ID NO: 14 - DNA, 1554 bp, unidentified
  source 1..1554: mol_type = genomic DNA; note = Pichia pastoris
atgcaattcaactgggatattaaaactgtggcaagtattttgtccgctctcacactagcacaagcaagtgatcaggaggctattgctccagaggactctcatgtcgtcaaattgactgaagccacttttgagtctttcatcaccagtaatcctcacgttttggcagagttttttgccccttggtgtggtcactgtaagaagttgggccctgaacttgtttctgctgccgagattttaaaggacaatgagcaggttaagattgctcaaattgattgtacggaggagaaggaattatgtcaaggctacgaaattaaagggtatcctactttgaaggtgttccatggtgaggttgaggtcccaagtgactatcaaggtcaaagacagagccaaagcattgtcagctatatgctaaagcagagtttaccccctgtcagtgaaatcaatgcaaccaaagatttagacgacacaatcgccgaggcaaaagagcccgtgattgtgcaagtactaccggaagatgcatccaacttggaatctaacaccacattttacggagttgccggtactctcagagagaaattcacttttgtctccactaagtctactgattatgccaaaaaatacactagcgactcgactcctgcctatttgcttgtcagacctggcgaggaacctagtgtttactctggtgaggagttagatgagactcatttggtgcactggattgatattgagtccaaacctctatttggagacattgacggatctaccttcaaatcatacgctgaagctaacatccctttagcctactatttctatgagaacgaagaacaacgtgctgctgctgccgatattattaaaccttttgctaaagagcaacgtggcaaaattaactttgttggcttagatgccgttaaattcggtaagcatgccaagaacttaaacatggatgaagagaaactccctctatttgtcattcatgatttggtgagcaacaagaagtttggagttcctcaagaccaagaattgacgaacaaagatgtgaccgagctgattgagaaattcatcgcaggagaggcagaaccaattgtgaaatcagagccaattccagaaattcaagaagagaaagtcttcaagctagtcggaaaggcccacgatgaagttgtcttcgatgaatctaaagatgttctagtcaagtactacgccccttggtgtggtcactgtaagagaatggctcctgcttatgaggaattggctactctttacgccaatgatgaggatgcctcttcaaaggttgtgattgcaaaacttgatcacactttgaacgatgttgacaacgttgatattcaaggttatcctactttgatcctttatccagctggtgataaatccaatcctcaactgtatgatggatctcgtgacctagaatcattggctgagtttgtaaaggagagaggaacccacaaagtggatgccctagcactcagaccagtcgaggaagaaaaggaagctgaagaagaagctgaaagtgaggcagacgctcacgacgagctttaa

SEQ ID NO: 15 - protein, 579 aa, unidentified
  source 1..579: note = Pichia pastoris
MASSSTSISLLLFVSFILLLVNSRAENASSGSDLDEELAFLAAEESKEQSHGGGSYHEEEHDHQHRDFENYDDLEQGGGEFHHGDHGYEEEPLPPVDEKDVAVLTKDNFTEFVGNNSFAMVEFYAPWCGACQALTPEYAAAATELKGLAALAKIDATEEGDLAQKYEIQGFPTVFLFVDGEMRKTYEGERTKDGIVTWLKKKASPSIHNITTKEEAERVLSAEPKLVFGFLNSLVGSESEELAAASRLEDDLSFYQTASPDIAKLFEIETQVKRPALVLLKKEEEKLARFDGNFTKTAIAEFVSANKVPLVINFTREGASLIFESSVKNQLILFAKANESEKHLPTLREVAKSFKGKFVFVYVQMDNEDYGEAVSGFFGVTGAAPKVLVYTGNEDMRKFILDGELTVNNIKTLAEDFLADKLKPFYKSDPLPENNDGDVKVIVGNNFDEIVLDESKDVLLEIYAPWCGHCQSFEPIYNKLGKYLKGIDSLVVAKMDGTSNEHPRAKADGFPTILFFPGGNKSFDPIAVDVDRTVVELYKFLKKHASIPFKLEKPATPEPVISTMKSDEKIEGDSSKDEL

SEQ ID NO: 16 - DNA, 1740 bp, unidentified
  source 1..1740: mol_type = genomic DNA; note = Pichia pastoris
atggcttcttcttctacttctatttctttgttgttgttcgtttctttcatcttgttgttggttaattctagagctgaaaacgcttcttctggttctgatttggatgaagaattggcttttcttgctgctgaagaatctaaagaacaatctcatggtggtggttcttatcatgaagaagaacatgatcatcaacatagagattttgaaaactacgatgatttggaacaaggtggtggtgaatttcatcatggtgaccatggttacgaagaagaaccattgccaccagttgatgaaaaagatgttgctgttttgactaaagataacttcactgaatttgtcggtaataactctttcgctatggttgaattttacgctccatggtgtggtgcttgtcaagctttgactcctgaatatgctgctgctgctactgaattgaaaggtttggctgctttggctaagattgatgctactgaagaaggtgacttggctcaaaagtatgaaattcaaggttttcctactgttttcttgtttgttgatggtgaaatgagaaagacttatgaaggtgaaagaactaaggatggtattgttacttggttgaaaaagaaagcttctccttctattcataacattactactaaggaagaggctgaaagagttttgtctgctgaaccaaagttggtttttggttttcttaactctttggttggttctgaatctgaagaattggccgctgcttctagattggaagatgatttgtctttttaccaaactgcttctcctgatattgctaaattgttcgaaattgaaacccaagttaagcgtcctgctttggttttgttgaaaaaggaagaagaaaagttggctagatttgatggtaattttactaagactgctatcgctgaatttgtttctgctaataaggttccattggttattaatttcaccagagaaggtgcttctttgattttcgaatcttctgttaagaaccaattgattttgttcgctaaagctaatgaatctgaaaagcatttgcctactttgagagaagttgctaagtctttcaaaggtaaattcgttttcgtttacgttcaaatggataatgaagattacggtgaagctgtttctggtttctttggtgttactggtgctgctccaaaggttttggtttatactggtaacgaagatatgagaaagttcattttggatggtgaattgactgttaacaatattaagactctggctgaagattttcttgctgataagttgaaaccattctacaagtctgatccattgcctgaaaacaacgatggtgacgttaaggttattgttggtaacaacttcgatgaaattgttttggatgaatctaaggatgttttgttggaaatctatgctccatggtgcggtcattgtcaatcttttgaaccaatctataacaagttgggtaaatacttgaagggtattgattctttggttgttgctaaaatggatggtacttctaacgaacatccaagagctaaagctgatggttttcctaccattttgtttttccctggtggtaataagtctttcgatcctattgctgttgatgttgatagaactgttgttgaattgtataagttcttgaagaagcatgcttctattcctttcaagttggaaaagccagctactccagaacctgttatttctactatgaagtctgatgaaaagatcgaaggtgactcttctaaggatgaattgtaa

SEQ ID NO: 17 - protein, 331 aa, unidentified
  source 1..331: note = Pichia pastoris
MPVDSSHKTASPLPPRKRAKTEEEKEQRRVERILRNRRAAHASREKKRRHVEFLENHVVDLESALQESAKATNKLKEIQDIIVSRLEALGGTVSDLDLTVPEVDFPKSSDLEPMSDLSTSSKSEKASTSTRRSLTEDLDEDDVAEYDDEEEDEELPRKMKVLNDKNKSTSIKQEKLNELPSPLSSDFSDVDEEKSTLTHLKLQQQQQQPVDNYVSTPLSLPEDSVDFINPGNLKIESDENFLLSSNTLQIKHENDTDYITTAPSGSINDFFNSYDISESNRLHHPAVMTDSSLHITAGSIGFFSLIGGGESSVAGRRSSVGTYQLTCIAIR

SEQ ID NO: 18 - DNA, 993 bp, unidentified
  source 1..993: mol_type = genomic DNA; note = Pichia pastoris
atgcccgtagattcttctcataagacagctagcccacttccacctcgtaaaagagcaaagacggaagaagaaaaggagcagcgtcgagtggaacgtatcctacgtaataggagagcggcccatgcttccagagagaagaaacgaagacacgttgaatttctggaaaaccacgtcgtcgacctggaatctgcacttcaagaatcagccaaagccactaacaagttgaaagaaatacaagatatcattgtttcaaggttggaagccttaggtggtaccgtctcagatttggatttaacagttccggaagtcgattttcccaaatcttctgatttggaacccatgtctgatctctcaacttcttcgaaatcggagaaagcatctacatccactcgcagatctttgactgaggatctggacgaagatgacgtcgctgaatatgacgacgaagaagaggacgaagagttacccaggaaaatgaaagtcttaaacgacaaaaacaagagcacatctatcaagcaggagaagttgaatgaacttccatctcctttgtcatccgatttttcagacgtagatgaagaaaagtcaactctcacacatttaaagttgcaacagcaacaacaacaaccagtagacaattatgtttctactcctttgagtcttccggaggattcagttgattttattaacccaggtaacttaaaaatagagtccgatgagaacttcttgttgagttcaaatactttacaaataaaacacgaaaatgacaccgactacattactacagctccatcaggttccatcaatgatttttttaattcttatgacattagcgagtcgaatcggttgcatcatccagcagtgatgacggattcatctttacacattacagcaggctccatcggctttttctctttgattggggggggggaaagttctgtagcagggaggcgcagttcagttggcacatatcagttgacatgcatagcgatcagg

SEQ ID NO: 19 - protein, 469 aa, unidentified
  source 1..469: note = Pichia pastoris
MGKGAIKEEESEKKRKTWRWPLATLVVVFLAVAVSSRTNSNVGFFFSDRNSCSCSLQKTGKYKGMIEDCCCDYETVDNLNTEVLNPLLQDLVTTPFFRYYKVKLWCDCPFWPDDGMCRLRDCSVCECPENEFPEPFKKPFVPGLPSDDLKCQEGKPQGAVDRTIDNRAFRGWVETKNPWTHDDDTDSGEMSYVNLQLNPERYTGYTGPSARRIWDSIYSENCPKYSSGETCPEKKVLYKLISGLHSSISMHIAADYLLDESRNQWGQNIELMYDRILRHPDRVRNMYFTYLFVLRAVTKATAYLEQAEYDTGNHAEDLKTQSLIKQLLYSPKLQTACPVPFDEAKLWQGQSGPELKQQIQKQFRNISALMDCVGCEKCRLWGKLQVQGLGTALKILFSVGNQDIGDQTLQLQRNEVIALVNLLNRLSESVKMVHDMSPDVERLMEDQIAKVSAKPARLRRIWDLAVSFW

SEQ ID NO: 20 - DNA, 1410 bp, unidentified
  source 1..1410: mol_type = genomic DNA; note = Pichia pastoris
atgggtaaaggtgctattaaggaagaagaatctgaaaagaagagaaaaacttggagatggcctttggctactttggttgttgttttcttggctgttgctgtttcttctagaactaactctaacgttggtttctttttctctgatagaaattcttgttcctgttctttgcaaaaaactggtaaatacaagggtatgattgaagattgttgttgtgattatgagactgttgataacttgaatactgaagttttgaaccctttgttgcaagatttggttactactccatttttcagatactacaaagttaagttgtggtgtgattgtccattctggccagatgatggtatgtgtagattgagagattgttctgtttgtgaatgtccagaaaacgaatttcctgaaccattcaaaaagcctttcgttcctggtttgccatctgatgatttgaaatgtcaagaaggtaaaccacaaggtgctgttgatagaactattgataacagagcttttagaggttgggttgaaactaaaaacccttggactcatgatgatgatactgattctggtgaaatgtcttatgttaatttgcaattgaacccagaaagatacactggttacactggtccttctgctagaagaatttgggattctatctattctgaaaactgtccaaagtactcttctggtgaaacttgtccagaaaagaaagttttgtataagttgatctccggtttgcattcttctatttctatgcatattgctgctgattatttgttggatgaatctagaaatcagtggggtcaaaacattgaattgatgtatgatagaatcctgagacatccagatagagttagaaatatgtatttcacttacctgttcgttttgagagctgttactaaagctactgcttatttggaacaagctgaatacgatactggtaaccatgctgaagatttgaaaactcaatctttgattaagcagttgttgtattctcctaaattgcaaactgcttgtccagttccttttgatgaagctaagttgtggcaaggtcaatctggtccagaattgaaacaacaaattcaaaaacagttcagaaacatctctgctttgatggattgtgttggttgtgaaaagtgtagattgtggggtaaattgcaagttcaaggtttgggtactgctttgaaaattttgttttctgttggtaaccaggatatcggtgaccaaactttgcaattgcaaagaaacgaagttattgctttggttaatttgttgaacagattgtctgaatctgttaagatggttcatgatatgtctccagatgttgaaagattgatggaagatcaaattgctaaagtttctgctaaacctgctagattgagaagaatttgggacttggctgtttctttctggtaa

SEQ ID NO: 21 - protein, 472 aa, unidentified
  source 1..472: note = Pichia pastoris
MAETDVGSVKGKEKGSGKRWILLIGAIAAVLLAVVVAVFLNTQNSSISEFTGKICNCRQAEQQKYIGIVEDCCCDYETVNRLNTEVLNPLLQDLVKTPFYRYFKVKLWCDCPFWPDDGMCRLRDCSVCECPESEFPEVFKKPLSQYNPVCQEGKPQATVDRTLDTRAFRGWTVTDNPWTSDDETDNDEMTYVNLRLNPERYTGYIGPSARRIWEAIYSENCPKHTSEGSCQEEKILYKLVSGLHSSISVHIASDYLLDEATNLWGQNLTLLYDRVLRYPDRVQNLYFTFLFVLRAVTKAEDYLGEAEYETGNVIEDLKTKSLVKQVVSDPKTKAACPVPFDEAKLWKGQRGPELKQQLEKQFRNISAIMDCVGCEKCRLWGKLQILGLGTALKILFTVNGEDNLRHNLELQRNEVIALMNLLHRLSESVKYVHDMSPAAERIAGGHASSGNSFWQRIVTSIAQSKAVSGKRS

SEQ ID NO: 22 - DNA, 1419 bp, unidentified
  source 1..1419: mol_type = genomic DNA; note = Pichia pastoris
atggctgaaactgatgttggttctgttaagggtaaagaaaagggttctggtaaaagatggattttgttgattggtgctattgctgctgttttgttggctgttgttgttgctgttttcttgaacactcaaaactcttctatttctgagtttactggtaaaatctgtaactgtagacaagctgaacaacaaaagtacattggtattgttgaagattgttgttgtgattatgagactgttaacagattgaacactgaagttttgaacccattgttgcaagatttggttaagactccattctacagatactttaaggttaagttgtggtgtgattgtcctttctggccagatgatggtatgtgtagattgagagattgttctgtttgtgaatgtccagaatctgaatttcctgaagttttcaagaaacctttgtctcaatataacccagtttgtcaagaaggtaaaccacaagctactgttgatagaactttggatactagagctttcagaggttggactgttactgataatccttggacttctgatgatgaaactgataacgatgaaatgacttatgttaacttgagattgaacccagaaagatacactggttatattggtccatctgctagaagaatttgggaagctatctattctgaaaattgtccaaaacatacctctgaaggttcttgtcaagaagaaaagattttgtataagctggtttctggtttgcattcttctatttccgttcatattgcttctgattacttgttggatgaagctactaacttgtggggtcaaaacttgactttgttgtatgatagagttttgagatacccagatagagttcaaaacttgtactttactttcttgttcgttttgagagctgttactaaagctgaagattacttgggtgaagctgaatacgaaactggtaacgttattgaagatttgaaaactaaatctctggtcaagcaagttgtttctgatccaaaaactaaggctgcttgtccagttccatttgatgaagctaagttgtggaagggtcaaagaggtccagaattgaagcaacaattggaaaagcaatttcgtaacatttctgctattatggattgtgttggttgtgaaaaatgtagattgtggggtaaattgcaaattttgggtttgggtactgctttgaaaattttgtttactgttaacggtgaggataatttgagacataacttggaattgcaaagaaacgaagttattgctttgatgaatttgttgcatagattgtctgaatctgttaaatacgttcatgatatgtctcctgctgctgaaagaattgctggtggtcatgcttcttctggtaattctttttggcaaagaattgttacttccattgctcaatctaaagctgtttctggtaaaagatcctaa

SEQ ID NO: 23 - protein, 191 aa, unidentified
  source 1..191: note = Pichia pastoris
MGEKPWQPLLQSFEKLSNCVQTHLSNFIGIKNTPPSSQSTIQNPIISLDSSPPIATNSSSLQKLPLKDKSTGPVTKEDLGRATWTFLHTLAAQYPEKPTRQQKKDVKELMTILSRMYPCRECADHFKEILRSNPAQAGSQEEFSQWLCHVHNTVNRSLGKLVFPCERVDARWGKLECEQKSCDLHGTSMDF

SEQ ID NO: 24 - DNA, 576 bp, unidentified
  source 1..576: mol_type = genomic DNA; note = Pichia pastoris
atgggtgaaaaaccatggcaaccattgttgcaatctttcgaaaagttgtctaattgtgttcaaactcatttgtctaacttcattggtattaagaacactccaccatcttctcaatctactattcaaaaccctattatctctttggattcttctccaccaattgctactaattcttcttctttgcaaaagttgcctttgaaggataagtctactggtccagttactaaggaagatttgggtagagctacttggacttttcttcatactttggctgctcaataccctgaaaaacctactagacaacaaaagaaagatgttaaggaattgatgactatcttgtctagaatgtatccatgtagagaatgtgctgatcatttcaaagaaattttgagatccaaccctgctcaagctggttctcaagaagaattttctcaatggttgtgtcatgttcataacactgttaatagatccttgggtaaattggttttcccttgtgaaagagttgatgctagatggggtaaattggaatgtgaacaaaaatcttgtgacttgcatggtacttctatggatttttaa

SEQ ID NO: 25 - protein, 205 aa, unidentified
  source 1..205: note = Pichia pastoris
MIKFNKRVATLTATLLSFIVLYTLFNSGARFANQLDQPVPLKTPELIIPNQSTKNDAPLPFMPKMANETLKAELGNASWKLFHTILARYPESPSENQKSTLNDYIYLFAQVYPCGDCARHFNLLLQKYPPQLSSRQVAAVWGCHIHNQVNKRLEKPQYDCSNILEDYDCGCGSDEKEVDDTLNNETMEHLQSIKITEKENEQFGR

SEQ ID NO: 26 - DNA, 615 bp, unidentified
  source 1..615: mol_type = genomic DNA; note = Pichia pastoris
atgataacattcaacaaacgaatagcaacattagcggcaacgttattttcattcattgtgctttatactctctttaacagtggtgctcaattttccaaccaactagatcagcctgttcccctcaaaactccagaactcatcataccgaatcagagtactgagaatgatccccctcttccattcatgccaaaaatggctaacgaaactttgaaagcagaacttggaaatgcttcctggaaactctttcacactattcttgctagatatcctgaatccccatcggagaatcaaaaatcaaccttaaatgactacatttatttgtttgcacaggtttatccatgtggagactgtgcaagacatttcaatttattgctgcagaaataccctccacaattgtcctcaagacaggtggctgcagtgtggggatgtcatattcacaatcaggtcaataagagattggagaaaccacaatacgactgctccaatattctagaggattacgattgtggatgtggctctgatgaaaaggaagtagatgacactctgaataacgaaacaatagaacacttgcaaagtatcaaaattactgaaaaagagagtgaacaatttggtcga

SEQ ID NO: 27 - protein, 527 aa, unidentified
  source 1..527: note = Pichia pastoris
MRIVRSLAVTITCYCITALANPQIPFDGNYTEITVPDTEVNIGQIVDINHEIKPKLVELVNTDFFKYYKLNLWKPCPFWNGDEGFCKYKDCSVDFITDWSQVPDIWQPDQLGKLGDNTVHKDKGQDENELSSNDYCALDKDDDEDLVYVNLIDNPERFTGYGGQQSESIWTAVYDENCFQPNEGSQLGQVEDLCLEKQIFYRLVSGLHSSISTHLTNEYLNLKNGEYEPNLKQFMIKVGYFTERIQNLHLNYVLVLKSLIKLQEYNVIENLPLDDSLKAGLSGLISQGAQNINQTDDYLFNEKVLFQNDQNDDLKNEFRDKFRNVTRLMDCVHCERCKLWGKLQTTGYGTALKILFDLKNPNDSINLKRVELVALVNTFHRLSKSVESIENFEKLYKIQPPTQDHPSPSSESLDVFDNEDEQNFFDSFSVDQTVTSSKEPPEEIKSKPVGKAEYKKTNSCPSSGSKSIKEAFHEELYAFIDAIGFILNSYRTLPKLLYTLFLVKSSELWDIFIGTQRHRDSTYRVDL

SEQ ID NO: 28 - DNA, 1598 bp, unidentified
  source 1..1598: mol_type = genomic DNA; note = Pichia pastoris
atgaggatagtaaggagcgtagctatcgcaatagcctgtcattgtataacagcgttagcaaaccctcaaatcccttttgacggcaactacaccgagatcatcgtgccagataccgaagttaacatcggacagattgtagatattaaccacgaaataaaacccaaactggtggaactggtcaacacagacttcttcaaatattacaaattaaacctatggaaaccatgtccgttttggaatggtgatgagggattctgcaagtataaggattgctctgttgactttatcactgattggtcccaggtgcctgatatctggcaaccagaccaattgggtaagcttggagataacacggtacataaggataagggccaagatgaaaatgagctgtcctcaaatgattattgcgctttggataaagacgacgatgaagatttagtatatgtcaatttgattgataaccctgaaagattcaccggttatggtggtcagcaatctgaatctatttggactgcggtctatgatgagaactgtttccagccgaatgaaggatcacaattgggtcaagttgaagacctctgtttggagaaacaaatcttttaccgattggtttctggtttgcattctagtatctccacccacctcacaaacgaatatctgaatttgaaaaatggagcatacgaaccaaatttgaaacagttcatgatcaaagttgggtattttactgaaagaatccaaaacttacatctcaattatgtccttgtattgaagtcactaataaagctacaagaatacaatgttatcgacaatctacctctcgatgactctttgaaagctggtcttagcggtttaatatctcaaggagcacagggtattaaccagagttctgatgattatctatttaacgagaaggttcttttccaaaatgaccaaaatgatgatttgaaaaatgaatttcgtgacaaattccgcaacgtgactagattaatggattgtgtccattgcgagagatgcaaattatggggaaaattgcaaactacagggtacgggactgcattgaagattctatttgatttgaagaatcctaatgactccatcaatttaaagagagttgagttagttgctctagtcaacacattccatagattgtccaaatctgttgaaagcattgaaaactttgaaaaactatataagattcaaccgccaacgcaggatcgtgcatcagcgtcgtccgaatccttaggccttttcgataacgaagatgaacaaaatctcctcaactcgttttcggttgatcaggcagtcatttcatcgaaagaggcaccagaagaaatcaaaagcaaacctgttggaaaagccgcatataaacaaaacagttgtccatcattgggttcaaaatctatcaaagaagcattccatgaagaacttcacgcatttattgatgcaattggatttatattgaactcttacaggactttgcccaagctgttgtacacacttttcctcgttaaatcatctgaattatgggacattttcattggcactcaaaggcaccgagataccacatatagagtagacttgtaagcggccgccagctt

SEQ ID NO: 29 - protein, 678 aa, unidentified
  source 1..678: note = Pichia pastoris
MLSLKPSWLTLAALMYAMLLVVVPFAKPVRADDVESYGTVIGIDLGTTYSCVGVMKSGRVEILANDQGNRITPSYVSFTEDERLVGDAAKNLAASNPKNTIFDIKRLIGMKYDAPEVQRDLKRLPYTVKSKNGQPVVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISNNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDYFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGTPPGGQGFDDDDGDFDYDYDYDHDEL

SEQ ID NO: 30 - DNA, 2037 bp, unidentified
  source 1..2037: mol_type = genomic DNA; note = Pichia pastoris
atgctgtcgttaaaaccatcttggctgactttggcggcattaatgtatgccatgctattggtcgtagtgccatttgctaaacctgttagagctgacgatgtcgaatcttatggaacagtgattggtatcgatttgggtaccacgtactcttgtgtcggtgtgatgaagtcgggtcgtgtagaaattcttgctaatgaccaaggtaacagaatcactccttcctacgttagtttcactgaagacgagagactggttggtgatgctgctaagaacttagctgcttctaacccaaaaaacaccatctttgatattaagagattgatcggtatgaagtatgatgccccagaggtccaaagagacttgaagcgtcttccttacactgtcaagagcaagaacggccaacctgtcgtttctgtcgagtacaagggtgaggagaagtctttcactcctgaggagatttccgccatggtcttgggtaagatgaagttgatcgctgaggactacttaggaaagaaagtcactcatgctgtcgttaccgttccagcctacttcaacgacgctcaacgtcaagccactaaggatgccggtctgatcgccggtttgactgttctgagaattgtgaacgagcctaccgccgctgcccttgcttacggtttggacaagactggtgaggaaagacagatcatcgtctacgacttgggtggaggaaccttcgatgtttctctgctttctattgagggtggtgctttcgaggttcttgctaccgccggtgacacccacttgggtggtgaggactttgactacagagttgttcgccacttcgttaagattttcaagaagaagcataacattgacatcagcaacaatgataaggctttaggtaagctgaagagagaggtcgaaaaggccaagcgtactttgtcttcccagatgactaccagaattgagattgactctttcgttgacggtatcgacttctctgagcaactgtctagagctaagtttgaggagatcaacattgaattattcaagaagacactgaaaccagttgaacaagtcctcaaagacgctggtgtcaagaaatctgaaattgatgacattgtcttggttggtggttctaccagaatcccaaaggttcaacaattattggaggattactttgacggaaagaaggcttctaagggaattaacccagatgaagctgtcgcatacggtgctgctgttcaggctggtgttttgtctggtgaggaaggtgtcgatgacatcgtcttgcttgatgtgaaccccctaactctgggtatcgagactactggtggcgttatgactaccttaatcaacagaaacactgctatcccaactaagaaatctcaaattttctccactgctgctgacaaccagccaactgtgttgattcaagtttatgagggtgagagagccttggctaaggacaacaacttgcttggtaaattcgagctgactggtattccaccagctccaagaggtactcctcaagttgaggttacttttgttttagacgctaacggaattttgaaggtttctgccaccgataagggaactggaaaatccgagtccatcaccatcaacaatgatcgtggtagattgtccaaggaggaggttgaccgtatggttgaagaggccgagaagtacgccgctgaggatgctgcactaagagaaaagattgaggctagaaacgctctggagaactacgctcattcccttaggaaccaagttactgatgactctgaaaccgggcttggttctaaattggacgaggacgacaaagagacattgacagatgccatcaaagataccctagagttcttggaagacaacttcgacaccgcaaccaaggaagaattagacgaacaaagagaaaagctttccaagattgcttacccaatcacttctaagctatacggtgctccagagggtggtactccacctggtggtcaaggttttgacgatgatgatggagactttgactacgactatgactatgatcatgatgagttgtaa

SEQ ID NO: 31 - protein, 568 aa, unidentified
  source 1..568: note = Pichia pastoris
MDLVKVGQSYVDKIVTDTGIKVLLLDDITSSIISLVSTQSELLNHQVYLIDKLENENRDTIKQLDCVCFLSVSEKTINLLVEELGAPKYKSYKLYFNNVVPNSFLERLAERDDLEMVDKVMELFLDYDILNKNLFSFKQLNIFNSIDAWNQQQFLLTLASLKSLCFSLQTNPIIRYESNSRMCSKLASDLSYEFGQSSKIMEKFPVNDIPPVLLILDRKNDPITPLLNPWTYQSMVHELLGIFNNTVDLTGTPSDLPPDLIKLVLNPSQDPFYAQSLYLNFGDLSDSIKTYVNEYKEKTVKHNSNELTDLNDMKHFLESFPEFKKLSNNISKHMGLITELDRKINENHLWQVSELEQSIAVNDNHNADLQELEKLLTSQEFKIANNLKVKLVCLYAIRYELHPNNQLPKMLSILLQQGVPEFEINTVNRMLKYSGSTKRLNDDSESSIFNQATNNLLQGFKQSHENDNIYMQHIPRLERVISKLVKNKLPTAHYPTLINDFLKKQRPVSDLNGARLQDIIIFFVGGVTYEEARIINNFNLVNKSTRIVIGGTTVHNTNSFMTQVLELE

SEQ ID NO: 32 - DNA, 1707 bp, unidentified
  source 1..1707: mol_type = genomic DNA; note = Pichia pastoris
atggacttggttaaggttggacaatcctacgtggataaaattgtcacagacacaggcattaaggttcttttattggatgatatcacttcttccataatttccctagtgagcacccaatcagaattgttgaaccatcaggtgtatttgatcgacaagttggagaacgagaatagagatacgataaagcaattggattgtgtgtgtttcctatcagtatcagaaaaaactataaacttgcttgttgaggaattaggtgctcccaaatacaaatcctacaagctctacttcaataatgtagttcccaactcattcttagagaggttggcggagagggacgatttggaaatggtcgataaggtcatggaattgttcctagattacgacattttgaacaagaacttgttttccttcaaacaactgaatattttcaattcaattgatgcttggaatcagcaacagtttctcttgactttagcaagcttgaaatcactctgcttctccttgcaaacgaatcctataatcaggtatgaatctaatagtcgaatgtgttctaagctagcttccgatttgtcatacgaatttgggcaaagttctaaaattatggaaaagttcccggtgaatgatatccctcctgtcctgttaattcttgaccgaaaaaacgacccaatcactccattattaaatccttggacttatcaatctatggtacacgagcttttaggaattttcaataatacggtggatttaacgggaactccttctgatctgcccccagacctaatcaaactggtattgaatccctctcaagatccattttatgctcagtctctatatttgaatttcggagacttgtccgatagtataaaaacatacgtaaacgagtacaaagaaaaaaccgtcaaacacaattctaatgaattgacagatttgaatgatatgaaacactttctggaatcttttccagagttcaaaaaactttcaaacaacatttccaaacacatgggcttgattacagaattagatagaaaaatcaacgaaaatcacttatggcaagtgagtgaattggaacaatccatagctgttaatgacaatcataatgctgaccttcaagaactagaaaagctgttgacatctcaagagttcaagattgccaacaacttaaaagttaaattagtatgtttgtatgccatacgatatgaacttcatcccaacaaccagcttccaaaaatgttgtcaatacttttacagcagggggtgccagagtttgaaataaatacagtcaacaggatgttgaaatactcgggaagtaccaaacgattgaatgatgactctgaatcttcgatatttaaccaggcaacaaataatctactgcaggggttcaaacaaagtcatgaaaacgacaatatttatatgcagcatattccaaggttggaaagagttatcagcaagttagtgaaaaataagctacccacagcgcattatccgactttaatcaatgattttttgaagaagcaacgccctgtttctgatctaaatggagccaggctgcaagatattattattttctttgttggtggagtcacttatgaagaggcccgaataattaacaatttcaatctggtgaacaagtctacgaggatagttatagggggaactacagtacacaacacgaatagttttatgactcaagttctagaattggagtaa

SEQ ID NO: 33 - protein, 645 aa, unidentified
  source 1..645: note = Pichia pastoris
MSFTTSLPSLRDRQIATLEKMLHLNEPIVDNGSDIQAELTWKVLILDSRSTAIVSSVLRVNDLLSSGITMHSNIRSKRAALPDVPVIYFVEPNAENINFIIDDLERDQYAHFYINFTSSLNRDLLEEFAKKVATIGKSYKIKQVYDQYLDYIVTEPNLFSLDLVNIYSQLNNPNSLEDEINKVADKISNGIFAAILTMNGIPTIRCCRGGPAELIASKLDQKLRDHVINTKSSASFTNSKLVLILLDRNIDLASMFAHSWIYQCMVSDVFELKRNTIKIPSQKPNESTKEYDIDPKDFFWAANNSLPFPDAVENVENELSRYKADAAELTRKTGVSSLQDIDPNAITDTTDIQLAVKSLPELAFRKSILDMHMKVLASLLQELESKSLDSYFEIEQNYKDPKNQKQFISILNNGNEHTLNDKLRTYIMLYLLTDLPGSFVEECEEYFKKNSAELGSLSYIKRAKEVIKLSNYELSMSIDASHSTTSGLVNEAQKSALFQGLSSKLYGLTDGGSRLTEGVGSLITGLKNLLPDKKQLPITNIVESIMEPSLATQESIKLTDDYLYFDPISTRGVHSKPPKRQQYNNSIVFVVGGGNYLEYQNLQEWVTKTNTSNVNGTKSVIYGSTSIVTANEFLKECSLLGAEAK

SEQ ID NO: 34 - DNA, 1878 bp, unidentified
  source 1..1878: mol_type = genomic DNA; note = Pichia pastoris
atgcttcatttgaatgagcccattgtggataatggttcagatatacaagcggagttaacatggaaggtactgattctggatagtaggagtactgcaattgtttcttctgttctgcgagttaatgacctgctttcttctggcatcactatgcatagcaatatcagatccaagagagcggctttgccagatgttcctgtcatttactttgttgaacctaatgcggaaaatatcaactttatcattgatgacttggaaagagatcagtacgctcatttttatatcaacttcacttccagtctaaatagggaccttttggaggagtttgctaagaaagtggctacgattggtaagtcctacaagattaaacaggtttatgatcagtacctcgattacattgtcactgaacccaacctgttctctttggacttggttaacatttactcgcagctaaataaccctaactcactggaagatgaaatcaataaagttgctgacaagatttccaatggtatattcgcagcaatcctaactatgaatggtatccctactattagatgttgcagaggaggtccagcagaactaatagcgtccaaactagatcagaagctacgtgatcatgttatcaatacaaagtcatctgcctctttcactaacagtaaattagtgcttatcctgctggatagaaacattgatttggcttccatgtttgctcattcatggatttatcaatgtatggtgagtgatgtttttgagttgaaaagaaatacaatcaaaattccctctcaaaagcccaatgaatctacgaaagaatatgatatcgacccaaaggattttttttgggcagccaacaacagtttgcccttccctgatgctgtagaaaatgtggagaacgaactttctagatacaaagcggatgctgcagagctaactagaaagactggggtttcttctcttcaagatattgatcccaatgcaattactgacaccacagatatacagcttgctgtgaagtctttacctgaattggcttttagaaaaagcatccttgatatgcacatgaaagtacttgcgtctttgctgcaagaactggaatcaaagtcattggattcatactttgaaattgaacaaaactacaaagatcccaaaaaccagaagcagtttatcagtatcctcaacaacgggaatgagcataccttgaacgacaaactgagaacctacatcatgttgtatctgttaacagacctcccagggtcgttcgttgaagaatgtgaagagtatttcaaaaagaactccgctgagcttggttcgttgagttatatcaagcgggcaaaagaggtgatcaagttgtctaattatgagttgtccatgtcaattgatgctagccactcgaccactagtggattggtgaatgaagctcaaaagtctgctttgttccaaggattgtcgtccaagctatatggattaacagatggtggtagtaggcttacagagggggtggggtcattaattactgggttgaaaaacttgctacccgacaagaaacaactgcctattaccaatattgttgaatcgataatggaaccaagtctggccactcaagagtcgataaaactaacggacgattacctatattttgaccctattagcacaagaggagttcactccaaaccacccaaaagacagcaatacaacaattctattgtgtttgttgtaggagggggcaactatttggagtaccaaaatttgcaagaatgggttacgaagaccaatactagcaacgtcaatggcactaagtctgtaatctacggtagtaccagtatcgtgaccgcgaacgagttcttgaaggagtgctccttgctcggtgccgaagcaaaataa

SEQ ID NO: 35 - protein, 161 aa, unidentified
  source 1..161: note = Pichia pastoris
MSSFYDLAPLDKKGEPFPFEQLKGKVVLIVNVASKCGFTPQYTELEKLYKDHKDEGLTIVGFPCNQFGHQEPGNDEEIGQFCQLNFGVTFPILKKIDVNGSEADPVYEFLKSKKSGLLGFKGIKWNFEKFLIDKQGNVIERYSSLTKPSSIESKIEELLKK

SEQ ID NO: 36 - DNA, 486 bp, unidentified
  source 1..486: mol_type = genomic DNA; note = Pichia pastoris
atgtcttcattttatgatctggccccattagataagaaaggcgaaccttttcctttcgaacaattaaaaggcaaagtggtgttgattgtgaatgttgcttctaagtgtgggtttactccacaatataccgagttggaaaagctctacaaagaccacaaggacgagggattgactattgtcggatttccctgtaaccagtttggtcatcaggaaccaggaaatgatgaagaaattggacagttttgccagttgaattttggtgtaactttcccaattctaaaaaagattgatgtcaacggttcggaagctgatcctgtttacgaatttctcaagtcaaaaaagtctggtctgctcggattcaaaggtattaagtggaactttgaaaaattcttgatcgataagcaaggaaacgttattgagagatattcgtccttgactaagccctcatcgatcgagtccaagattgaagaactattaaagaaataa

SEQ ID NO: 37 - protein, 85 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 38 - DNA, 255 bp, synthetic construct
  source 1..255: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaga

SEQ ID NO: 39-40 - skipped (2 entries)

SEQ ID NO: 41 - protein, 4 aa, synthetic construct
KREA

SEQ ID NO: 42 - DNA, 12 bp, synthetic construct
  source 1..12: mol_type = other DNA
aagcgagaagcc

SEQ ID NO: 43 - protein, 12 aa, synthetic construct
KREAEAEAEAEA

SEQ ID NO: 44 - DNA, 36 bp, synthetic construct
  source 1..36: mol_type = other DNA
aagcgagaagccgaagcagaagcagaagcagaagcg

SEQ ID NO: 45 - protein, 6 aa, synthetic construct
KREAEA

SEQ ID NO: 46 - DNA, 18 bp, synthetic construct
  source 1..18: mol_type = other DNA
aagcgagaagccgaagca

SEQ ID NO: 47 - protein, 8 aa, synthetic construct
KREAEAEA

SEQ ID NO: 48 - DNA, 24 bp, synthetic construct
  source 1..24: mol_type = other DNA
aagcgagaagcagaagcagaagcg

SEQ ID NO: 49 - protein, 10 aa, synthetic construct
KREAEAEAEA

SEQ ID NO: 50 - DNA, 30 bp, synthetic construct
  source 1..30: mol_type = other DNA
aagcgagaagcagaagcagaagcagaagcg

SEQ ID NO: 51 - protein, 490 aa, synthetic construct
MRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 52 - DNA, 1470 bp, synthetic construct
  source 1..1470: mol_type = other DNA
atgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgct

SEQ ID NO: 53 - protein, 2056 aa, synthetic construct
MRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 54 - DNA, 6168 bp, synthetic construct
  source 1..6168: mol_type = other DNA
atgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctacttttgaaatcgttaacagatgttcttacactgtttgggctgctgcttctaaaggtgacgctgctttggatgctggtggtagacaattgaatagtggtgaatcttggactattaatgttgaaccaggtactaatggtggtaaaatttgggctagaactgattgttacttcgatgattctggttctggtatttgtaaaactggtgactgtggtggtttgttgagatgtaaaagattcggtagaccacctactactttggctgaattttctttgaaccaatacggtaaagattacattgatatctctaacatcaagggttttaacgttcctatggatttctctcctactactagaggttgtagaggtgttagatgtgctgctgatattgttggtcaatgtcctgctaagttgaaagctcctggtggtggttgtaacgatgcttgtactgtttttcaaacttctgaatattgctgtactactggtaaatgtggtccaactgaatactctagattcttcaaaagattgtgtcctgatgctttttcttacgttttggataaaccaactactgttacttgtcctggttcttctaattatagagttactttctgtccaactgct

SEQ ID NO: 55 - protein, 490 aa, synthetic construct
MRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 56 - DNA, 1470 bp, synthetic construct
  source 1..1470: mol_type = other DNA
atgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttttgtcctactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgct

SEQ ID NO: 57 - protein, 2056 aa, synthetic construct
MRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTAKREAEAEAEAEAGATNFSLLKLAGDVELNPGPMRQVWFSWIVGLFLCFFNVSSAATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 58 - DNA, 6168 bp, synthetic construct
  source 1..6168: mol_type = other DNA
atgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttttgtcctactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgctaagcgagaagccgaagcagaagcagaagcagaagcgggagcaactaatttcagtctcctgaagttggccggtgacgttgaactgaaccctggaccgatgaggcaggtttggttctcttggattgtgggattgttcctatgttttttcaacgtgtcttctgctgctactttcgaaattgttaatagatgttcttacaccgtttgggctgctgcttctaagggtgacgctgctttggatgctggtggtagacaattgaatagtggtgaatcttggactattaacgttgaacctggtactaagggtggtaaaatttgggctagaactgattgttacttcgatgattctggtagaggtatttgtagaactggtgactgtggtggtttgttgcaatgtaaaagatttggtagaccaccaactactttggctgaattttctttgaatcaatacggtaaagactacattgatatctctaatatcaagggtttcaacgttccaatggatttctctcctactactagaggttgtagaggtgttagatgtgctgctgatattgttggtcaatgtccagctaaattgaaagctccaggtggtggttgtaacgatgcttgtactgtttttcaaacttctgaatactgttgtactactggtaaatgtggtcctactgaatattctagatttttcaaaagactgtgcccagatgctttttcttacgttttggataaacctactactgttacttgtccaggttcttctaactatagagttactttctgtccaactgct